(19) United States
(12) Patent Application Publication
(10) Pub. No.: US 2025/0375383 A1
(43) Pub. Date: Dec. 11, 2025

(54) Structured solid dosage form

(71) Applicant: Aron H. Blaesi, Cambridge, MA (US)

(72) Inventors: Aron H. Blaesi, Cambridge, MA (US); Nannaji Saka, Cambridge, MA (US)

(73) Assignee: Aron H. Blaesi, Cambridge, MA (US)

(21) Appl. No.: 19/301,977

(22) Filed: Aug. 16, 2025

Related U.S. Application Data
(63) Continuation of application No. 19/074,409, filed on Mar. 9, 2025.
Application No. 19/074,409 is a continuation of application No. 17/367,344, filed on Jul. 3, 2021, now abandoned.
Application No. 17/367,344 is a continuation-in-part of application No. 16/916,208, filed on Jun. 30, 2020, now Pat. No. 11,478,427.

(51) Int. Cl. A61K 9/48; A61K 31/192

(52) U.S. Cl. CPC A61K 9/4808; A61K 9/4866; A61K 31/192

(57) ABSTRACT

At present, the most prevalent pharmaceutical dosage forms, the oral immediate-release tablets and capsules, are porous solids of compacted drug and excipient powders. Upon ingestion, physiological fluid percolates the open pores, and the dosage form disintegrates and the drug dissolves. Because the pores in the compacted solids are not well connected, however, fluid percolation generally is not uniform, and the drug release rate is difficult to predict and control. To overcome such limitations, therefore, herein a structured solid dosage form is disclosed. The structured solid dosage form comprises a structural assembly of one or more repeatably arranged, extruded structural elements. The elements comprise segments separated and spaced from adjoining segments by free spacings defining one or more substantially interconnected free spaces, or channels, in the dosage form through which a physiological fluid may percolate. The disclosed dosage form enables more predictable drug release rates, and can be readily manufactured by 3D-printing.

### CROSS-REFERENCE TO RELATED APPLICATIONS

This application is a continuation of, and incorporates herein by reference in its entirety, the U.S. application Ser. No. 19/074,409 filed on Mar. 9, 2025, and titled “Structured solid dosage form”, which is a continuation of the U.S. application Ser. No. 17/367,344 filed on Jul. 3, 2021, and titled “Structured solid dosage form”, which is a continuation-in-part of the U.S. application Ser. No. 16/916,208 filed on Jun. 30, 2020, and titled “Dosage form comprising structural framework of two-dimensional elements”, which is a continuation-in-part of the U.S. application Ser. No. 15/964,063 filed on Apr. 26, 2018, and titled “Dosage form comprising two-dimensional structural elements”, which is a continuation-in-part of the International Application No. PCT/US2016/058935 filed on Oct. 26, 2016, and titled “Solid Dosage Form for Immediate Drug Release and Apparatus and Method for Manufacture thereof”, which claims priority to and the benefit of the U.S. Provisional Application No. 62/246,470 filed Oct. 26, 2015, the U.S. Provisional Application No. 62/360,546 filed on Jul. 7, 2016, and the U.S. Provisional Application No. 62/377,068 filed on Aug. 19, 2016. All foregoing applications are hereby incorporated by reference in their entirety.

U.S. application Ser. No. 17/367,344 also is a continuation-in-part of the U.S. application Ser. No. 15/482,776 filed on Apr. 9, 2017, and titled “Fibrous dosage form”, which claims priority to and the benefit of the U.S. Provisional Application No. 62/446,431 filed on Jan. 14, 2017, and the U.S. Provisional Application No. 62/468,888 filed on Mar. 8, 2017. All foregoing applications are hereby incorporated by reference in their entirety.

### FIELD OF THE INVENTION

This invention relates generally to microstructures, compositions, and methods for drug release. In certain embodiments, the invention relates to dosage forms comprising a structural assembly of one or more repeatably arranged, drug-containing structural elements.

### BACKGROUND OF THE INVENTION

The most prevalent pharmaceutical dosage forms at present, the oral immediate-release tablets and capsules, are porous solids consisting of compacted drug and excipient particles. Upon ingestion, the gastrointestinal fluid percolates the open pores, and interdiffuses with the water-soluble excipient. The inter-granular bonds are then severed, the granules are released into the surrounding fluid, and dissolve. The large surface area-to-volume ratio of the small granules promotes rapid drug dissolution, and enables that a large fraction of the ingested drug is absorbed by the blood stream as detailed in the commonly owned references “Remington's Pharmaceutical Sciences XVIII”, A. R. Gennaro (ed.), Mack Publishing, Easton, PA, 1990; and M. E. Aulton, K. M. G. Taylor, “Aulton's pharmaceutics: The design and manufacture of medicines”, fourth edition, Churchill Livingstone, London, UK, 2013, and others.

Because the pores in the compacted particulate dosage forms are not well connected, however, fluid percolation generally is not uniform; hence numerous excipients (˜5-10) and multiple statistical process steps are typically required to ensure that the dosage form meets the specifications. As a result, dosage form development and manufacture are both resource-intensive and time-consuming. Moreover, addition of such common excipients as lactose, starch, sugars, polyols, and so on, is undesirable because they cause allergies or other adverse effects in some patients. For further details related to the manufacture and properties of particulate dosage forms, see, e.g., Y. Qiu, L. Lirong, G. Zhang, Y. Chen, and W. Porter, “Developing oral solid dosage forms: pharmaceutical theory and practice”, Academic Press, Burlington, MA, 2008; F. Muzzio, T. Shinbrot, B. J. Glasser, “Powder technology in the pharmaceutical industry: the need to catch up fast”, Powder Technol. 124, 2002, 1-7; and S. Yassin, D.J. Goodwin, A. Anderson, J. Sibik, D. I. Wilson, L. F. Gladden, J. A. Zeitler, “The disintegration process in microcrystalline cellulose based tablets, part 1: Influence of temperature, porosity and superdisintegrants”, J. Pharm. Sci. 104, 2015, pp. 3440-3450. For further details related to adverse effects of commonly used excipients, see, e.g., G. Pifferi, P. Restani, “The safety of pharmaceutical excipients”, Il Farmaco 58, 2003, 541-550; and D. Reker, S. M. Blum, C. Steiger, K. E. Anger, J. M. Sommer, J. Fanikos, G. Traverso, ““Inactive” ingredients in oral medications”, Sci. Trans. Med. 11, 2019, 6753.

More predictable dosage form development and manufacture could be achieved by liquid-based processing of the dosage forms, as the streamlines in laminar flow follow known pathways and the flow rates can be calculated from “constitutive” models. However, the solidification of a melt or the drying of a paste generally yields a non-porous (or minimally-porous), solid microstructure. Because the specific surface area of such non-porous dosage forms is small, their disintegration rate is much smaller than that of the compacted particulate forms. As a result, the non-porous dosage forms generally are not suited for immediate drug release.

To overcome the above and other limitations, herein a solid dosage form with substantially interconnected void space, or channels, in a controlled microstructure is disclosed. The disclosed dosage form enables more predictable drug release rates, and can be deterministically manufactured by an efficient 3D-printing process.

### SUMMARY OF THE INVENTION

More specifically, in a first aspect, the present invention provides a pharmaceutical solid dosage form comprising a drug-containing solid having an outer surface and an internal structure contiguous with and terminating at said outer surface; said internal structure comprising a continuous structural assembly of one or more repeatably arranged, extruded structural elements; said extruded structural elements comprising at least one pharmaceutically active ingredient and at least a hydrophilic excipient; said extruded structural elements further comprising segments separated and spaced from adjoining segments by free spacings, said free spacings defining one or more substantially interconnected free spaces through the drug-containing solid; wherein the one or more extruded structural elements are so arranged that an average ‘free spacing’ between segments is greater than 1 μm; and upon immersion of said drug-containing solid in a physiological fluid under physiological conditions, said drug-containing solid comprises a disintegration time of less than 45 minutes.

In some embodiments, average thickness of the one or more elements is no greater than 1 mm.

In some embodiments, average thickness of the one or more elements is in the range between 5 μm and 2 mm.

In some embodiments, the thickness of the one or more elements is precisely controlled.

In some embodiments, average ‘free spacing’ between segments is in the range between 1 μm and 5 mm.

In some embodiments, the ‘free spacing’ between segments of the one or more elements is precisely controlled.

In some embodiments, at least one structural element comprises a fiber.

In some embodiments, at least one structural element comprises a sheet.

In some embodiments, at least one structural element comprises a bead that is bonded to another structural element.

In some embodiments, upon immersion of the drug-containing solid in a physiological fluid under physiological conditions, said physiological or body fluid percolates a substantially interconnected free space.

In some embodiments, no more than 15 walls must be ruptured to obtain an interconnected, continuous free space from a surface of the drug-containing solid to any point in the interior.

In some embodiments, the free space is contiguous.

In some embodiments, at least a structural element or a segment thereof is bonded to another structural element or another segment of said structural element.

In some embodiments, at least a structural element or a segment thereof is bonded to another structural element or another segment of said structural element by solidification of a fluidic contact between said elements or segments.

In some embodiments, at least one excipient comprises a solubility no less than about 10 g/l in a physiological or body fluid under physiological conditions.

In some embodiments, at least one excipient is swellable by a body fluid, and wherein an effective diffusivity of water in said swellable excipient is greater than 1×10⁻¹¹ m²/s.

In some embodiments, the swellable excipient comprises a viscosity less than 500 Pa·s upon absorption of a physiological fluid under physiological conditions.

In some embodiments, at least one hydrophilic excipient is selected from the group comprising polyethylene glycol (PEG), polyethylene oxide, polyvinylpyrrolidone (PVP), PEG-PVP copolymer, poloxamer, lauroyl macrogol-32 glycerides, polyvinylalcohol (PVA), PEG-PVA copolymer, polylactic acid, polyvinylacetate phthalate, polymethacrylates (e.g., poly (methacrylic acid, ethyl acrylate) 1:1, or butylmethacrylat-(2-dimethylaminoethyl) methacrylat-methylmathacrylat-copolymer), gelatin, cellulose or cellulose derivatives (e.g., microcrystalline cellulose, hydroxypropyl cellulose, hydroxyethyl cellulose, methyl cellulose, hydroxypropyl methyl ether cellulose, or hydroxypropyl methylcellulose), starch, polylactide-co-glycolide, polyvinyl caprolactam-polyvinyl acetate-polyethylene glycol graft copolymer, lactose, starch derivatives (e.g., pregelatinized starch or sodium starch glycolate), chitosan, pectin, acrylic acid crosslinked with allyl sucrose or allyl pentaerythritol (e.g., carbopol), and polyacrylic acid.

In some embodiments, a free space comprises a matter selected from the group comprising gas, liquid, or solid, and wherein said matter is partially or entirely removed upon contact with a physiological or body fluid under physiological conditions.

In some embodiments a free space comprises at least a gas.

In some embodiments, a free space comprises at least a gas, and wherein the gas comprises at least one of air, nitrogen, CO₂, argon, or oxygen.

In some embodiments, upon immersion of said drug-containing solid in a physiological fluid under physiological conditions, said drug-containing solid comprises a disintegration time of less than 30 minutes.

In a second aspect, the invention herein provides a pharmaceutical solid dosage form comprising a drug-containing solid comprising a continuous structural assembly of one or more repeatably arranged, extruded structural elements; said extruded structural elements having an average thickness no greater than 1 mm; said extruded structural elements further comprising at least one pharmaceutically active ingredient and at least a hydrophilic excipient; said extruded structural elements further comprising segments separated and spaced from adjoining segments by free spacings, said free spacings definining one or more substantially interconnected free spaces through the drug-containing solid; wherein the one or more extruded structural elements are so arranged that an average ‘free spacing’ between segments is greater than 1 um; at least one free space comprises at least a gas; and upon immersion of said drug-containing solid in a physiological fluid under physiological conditions, said drug-containing solid comprises a disintegration time of less than about 30 minutes.

In a third aspect, the invention herein provides a pharmaceutical solid dosage form comprising a drug-containing solid comprising a continuous structural assembly of one or more repeatably arranged, extruded fibers with average fiber thickness no greater than 1 mm; said extruded fibers comprising at least one pharmaceutically active ingredient and at least a hydrophilic excipient, said hydrophilic excipient having a solubility in a physiological fluid under physiological conditions greater than 1 g/l; said extruded fibers further comprising segments separated and spaced from adjoining segments by free spacings, said free spacings definining one or more substantially interconnected free spaces through the drug-containing solid; wherein the one or more extruded fibers are so arranged that an average ‘free spacing’ between segments is greater than 1 um; at least one free space comprises at least a gas; and upon immersion of said drug-containing solid in a physiological fluid under physiological conditions, said drug-containing solid comprises a disintegration time of less than about 30 minutes.

Elements of embodiments described with respect to one aspect of the invention can be applied with respect to another aspect. By way of example but not by way of limitation, certain embodiments of the claims described with respect to the first aspect can include features of the claims described with respect to the second or third aspect, and vice versa.

This invention may be better understood by reference to the accompanying drawings, attention being called to the fact that the drawings are primarily for illustration, and should not be regarded as limiting. The scope of the invention is limited only by the claims and not by the drawings or description herein.

### BRIEF DESCRIPTION OF THE DRAWINGS

The objects, embodiments, features, and advantages of the present invention are more fully understood when considered in conjunction with the following accompanying drawings:

FIG. 1 schematically presents non-limiting microstructures of structured dosage forms according to this invention;

FIG. 2 presents additional schematics of dosage form microstructures according to this invention;

FIG. 3 schematically shows non-limiting disintegration processes of single elements (e.g. fibers) in both stagnant (not stirred) and stirred dissolution media;

FIG. 4 schematically presents the time-dependent conversion of a fibrous structure into a polymer-dissolution fluid solution after immersion of the fibrous structure in a stagnant dissolution fluid;

FIG. 5 illustrates schematics of fluid flow around and through a fibrous dosage

form in a stirred dissolution fluid;

FIG. 6 presents a non-limiting example of percolation of dissolution medium into an interconnected free space;

FIG. 7 shows schematics of the microstructure of solid dosage forms according to this invention to illustrate the ‘effective free spacing’ between adjoining elements or segments;

FIG. 8 illustrates a schematic of the contact angle of a fluid droplet on a surface;

FIG. 9 depicts a schematic diagram of the microstructure of solid dosage forms according to this invention to illustrate the number of walls that must be ruptured to obtain an interconnected cluster of free space that extends from the outer surface of the drug-containing solid to a point in the interior;

FIG. 10 schematically presents three elements of different thicknesses;

FIG. 11 is a schematically shows the microstructure of a coated solid dosage form according to this invention;

FIG. 12 presents a schematic of a dosage form comprising at least two drug-containing solids;

FIG. 13 is a schematic of a process and apparatus to manufacture the structured dosage forms disclosed herein;

FIG. 14 depicts scanning electron microscopy (SEM) images of dosage forms according to this invention;

FIG. 15 displays disintegration of melt-processed fibers in both stagnant and stirred dissolution fluid;

FIG. 16 presents disintegration of melt-processed dosage forms according to this invention in stirred dissolution fluid;

FIG. 17 shows disintegration of wet-processed fibers in both stagnant and stirred dissolution fluid;

FIG. 18 presents disintegration of wet-processed dosage forms according to this invention in stirred dissolution fluid;

FIG. 19 displays the results of the fraction of drug dissolved versus time of melt-processed dosage forms according to this invention;

FIG. 20 shows the results of the fraction of drug dissolved versus time of wet-processed dosage forms according to this invention;

FIG. 21 presents the shear viscosity of water-excipient solutions versus weight fraction of the polymeric excipient (PEG 35k);

FIG. 22 shows the results of shear viscosity measurements of additional water-excipient solutions versus weight fraction of the polymeric excipient. Polyvinyl alcohol-polyethylene glycol graft copolymer 3:1 with a molecular weight of 45,000 Daltons (tradename: Kollicoat IR) was the excipient in this case; and

FIG. 23 presents schematics of excipient polymer molecules solvated by a dissolution medium at a polymer concentration, cₚ, of (a) cₚ<cₚ*(or wₚ<wₚ*), (b) cₚ*≤cₚ≤cₚ**(or wₚ*≤wₚ≤wₚ**),and (c) cₚ>cₚ**(or wₚ>wₚ**). cₚ* is the disentanglement concentration of the polymer, cₚ** the polymer concentration at the transition between semi-dilute and concentrated solution, wₚ* the weight fraction of the polymer at disentanglement, and wₚ** the weight fraction of the polymer at the transition between semi-dilute and concentrated solution.

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

Moreover, in the disclosure herein, the terms “active ingredient”, “active ingredients”, “one or more active ingredients”, “active pharmaceutical ingredient”, “drug”, “one or more drugs”, and so on, are used interchangeably. As used herein, an “active ingredient” or “active agent” refers to an agent whose presence or level correlates with elevated level or activity of a target, as compared with that observed absent the agent (or with the agent at a different level). In some embodiments, an active ingredient is one whose presence or level correlates with a target level or activity that is comparable to or greater than a particular reference level or activity (e.g., that observed under appropriate reference conditions, such as presence of a known active agent, e.g., a positive control).

Furthermore, in the context of the invention herein, a structural assembly of drug-containing elements comprises a drug-containing structure of elements (e.g., an assemblage, a framework, a three dimensional framework, a network, an arrangement, etc. of one or more drug-containing elements) that extends over a length, width, and thickness greater than the thickness of at least one element. This includes, but is not limited to structural assemblies of elements that extend over a length, width, and thickness greater than twice the thickness of at least one element, or greater than three times the thickness of at least one element, or greater than four times the thickness of at least one element, or greater than five times the thickness of at least one element, or greater than six times the thickness of at least one element, or greater than seven times the thickness of at least one element.

As used herein, the terms “element”, “elements”, “one or more drug-containing elements”, “drug-containing elements”, “structural elements”, “drug-containing structural elements”, and so on are used interchangeably. They are understood as the solid, drug-containing building blocks (e.g., the structural elements) that make up part of or the entire dosage form structure (e.g., the structural assembly of elements). Generally, the thickness of an element is much smaller than the thickness of the solid dosage form it forms.

As used herein, a two-dimensional structural element is referred to as having a length and width much greater than its thickness. In the present disclosure, the length and width of a two-dimensional structural element generally are greater than 2 times its thickness. An example of such an element is a “sheet”. A one-dimensional structural element is referred to as having a length much greater than its width and/or thickness. In the present disclosure, the length of a one-dimensional structural element generally is greater than 2 times its width and thickness. An example of such an element is a “fiber”. A zero-dimensional structural element is referred to as having a length and width of the order of its thickness. In the present disclosure, the length and width of a zero-dimensional structural element generally are no greater than 2 times its thickness. Furthermore, the thickness of a zero-dimensional element is less than 2.5 mm. Examples of such zero-dimensional elements are “particles” or “beads” and include polyhedra, spheroids, ellipsoids, or clusters thereof.

As used herein the term “extruded structural element” generally refers to an element with fixed cross section along its length. Such “extruded structural elements” typically form a single solid matrix (e.g., a continuous matrix or a connected solid matrix or a connected solid structure) through their thickness. Generally, moreover, such extruded structural elements are prepared by extrusion of the element formulation through a die or nozzle with a specific cross section.

Generally, as used herein, one or more extruded structural elements are considered “repeatably arranged” if the microstructural features, such as the position of elements, the spacing between elements, and so on, are “precisely controlled” or reproducible within a tight margin. For further definitions of the term “precisely controlled”, see, e.g., paragraph 146 of the specification herein. Generally, moreover, a structural assembly of repeatably arranged elements comprises an ordered or partially ordered structure.

Furthermore, as used herein, the term “segment” refers to a fraction of an element along the length and/or width of said element.

In the context of the invention disclosed herein, drug release from a solid element (or a solid dosage form, or a solid matrix, or a drug-containing solid) refers to the conversion of drug (e.g., one or more drug particles, or drug molecules, or clusters thereof, etc.) that is/are embedded in or attached to the solid element (or the solid dosage form, or the solid matrix, or the drug-containing solid) to drug in a dissolution medium. If the drug is embedded in a polymeric excipient or matrix, the drug may be released from said polymeric matrix as soon as said polymeric matrix has converted to a dilute solution (e.g., a liquid in which the excipient concentration is smaller than its solubility or “interfacial concentration”).

Similarly, in the invention disclosed herein, a polymeric excipient matrix may be considered disintegrated if said polymeric matrix has converted to a gel with polymer concentration smaller than the “interfacial concentration” (e.g., as soon as the polymer has converted to a dilute solution).

In this application, the term “interfacial concentration” is referred to as the polymer concentration which separates the “solid” and “liquid” regions of a polymer eroding into a dissolution medium. It is typically of the order of the disentanglement concentration, cₚ*, of said polymer in a dissolution medium (or of the order of the solubility of said polymer in a dissolution medium).

As used herein, the terms “dissolution medium”, “physiological/body fluid”, “dissolution fluid”, “medium”, “fluid”, and “penetrant” are used interchangeably. They are understood as any fluid produced by or contained in a human body under physiological conditions, or any fluid that resembles a fluid produced by or contained in a human body under physiological conditions. Examples include, but are not limited to: water, saliva, stomach fluid, gastrointestinal fluid, saline, etc. at a temperature of 37° C. and a pH value adjusted to the specific physiological condition.

Finally, as used herein, free space may be considered “substantially interconnected” if it extends over a length greater or far greater than the average thickness of one or more elements. This includes, but is not limited to free space extending over a length greater than two times the average thickness of one or more elements, or free space extending over a length greater than three times the average thickness of one or more elements, or free space extending over a length greater than four times the average thickness of one or more elements, or free space extending over a length greater than five times the average thickness of one or more elements. Also, in some embodiments, free space may be considered “substantially interconnected” if it extends over a length greater than one third of the thickness of the drug-containing solid. This includes, but is not limited to free space that extends over a length greater than half the thickness of the drug-containing solid, or at least equal to about a thickness of the drug-containing solid.

### DETAILED DESCRIPTION OF THE INVENTION

### Dosage Form Structures

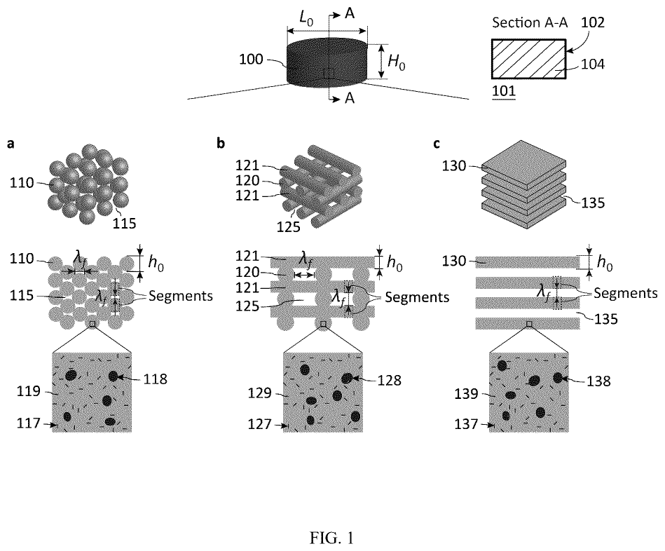

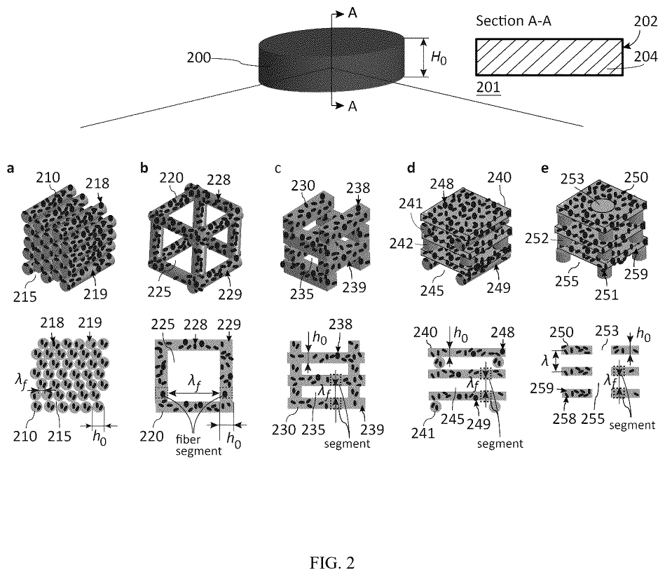

FIGS. 1 and 2 present non-limiting examples of pharmaceutical dosage forms 100, 200 comprising a drug-containing solid 101, 201 having an outer surface 102, 202 and an internal structure 104, 204 contiguous with and terminating at said outer surface 102, 102. The internal structure 204, 304 comprises a continuous structural assembly of one or more repeatably arranged, extruded structural elements 110, 120, 130, 210, 220, 230, 240, 250. The extruded structural elements 110, 120, 130, 210, 220, 230, 240, 250 comprise at least a pharmaceutically active ingredient 117, 118, 127, 128, 137, 138, 218, 228, 238, 248, 258 and at least an excipient 119, 129, 139, 219, 229, 239, 249, 259. The extruded structural elements 110, 120, 130, 210, 220, 230, 240, 250 further comprise segments separated and spaced from adjoining segments by free spacings, λ_{f}, defining one or more substantially interconnected free spaces 115, 125, 135, 215, 225, 235, 245, 255 (e.g., channels) through or within the drug-containing solid 101, 201.

The one or more extruded structural elements 110, 120, 130, 210, 220, 230, 240, 250 generally have at least one dimension (e.g., a length, width, or thickness) substantially smaller than a dimension (e.g., a length, width, or thickness) of the drug-containing solid 101, 201 or the dosage form 100, 200. Thus, the one or more extruded structural elements 110, 120, 130, 210, 220, 230, 240, 250 can be particles or beads 110 or “0-dimensional elements” with thickness, width, and length of the same order of magnitude, and substantially smaller than a dimension of the drug-containing solid 101, 201 or dosage form 100, 200. They can also be fibers 120, 210, 220, 230 or “1-dimensional elements” with thickness and width smaller than the length. The thickness and the width of a fiber or 1-dimensional element 120, 210, 220, 230 may further be smaller than a dimension of the drug-containing solid 101, 201 or dosage form 100, 200. They can further be planes or sheets 130, 240, 250 or “2-dimensional elements” with thickness smaller than the width and length. The thickness of a sheet or 2-dimensional element 130, 240, 250 may further be smaller than a dimension of the drug-containing solid 101, 201 or dosage form 100, 200.

The one or more extruded structural elements 110, 120, 130, 210, 220, 230, 240, 250 may be arranged (e.g., structured or oriented) in a variety of ways, ranging from random (e.g., disordered) to partially regular (e.g., partially ordered) to regular (e.g., ordered or not random). The ordered or partially ordered structures, however, with repeatably arranged elements are generally preferred herein. A few non-limiting examples of such structures are given below.

FIG. 1a shows a dosage form 100 or drug-containing solid 101 with a cubic (e.g., a body centered cubic) lattice structure of particles or beads 110. The particles or beads 110 are separated from adjoining particles or beads 110 by free spacings, is defining free spaces 105 in the drug-containing solid 101 or dosage form 100. The free spaces 105 are intrinsically connected in this structure; thus the free space 105 is contiguous. A primary structural variable that may be altered in this arrangement (or structural assembly) for tailoring the properties of the drug-containing solid 101 or dosage form 100 is the thickness, or diameter, ho, of the particles or beads 110.

FIG. 1b shows a dosage form 100 or drug-containing solid 101 with cross-ply arrangement (or structure) of fibers 120 with circular cross section. The fibers 120 in a plane or layer are oriented in one direction but the fibers 121 in the planes or layers above and below are oriented transversely, or at an angle. This arrangement (or structural assembly, or three dimensional structural network) provides control of two structural variables essential for tailoring the properties of the dosage form 100 or drug-containing solid 101: the fiber diameter, D_{f}=2R, (or the average fiber thickness, h₀) and the inter-fiber spacing, λ, in a plane (or alternatively the free spacing, λ_{f}). Moreover, the free spaces 125 around the fibers 120, 121 are intrinsically connected in this arrangement; thus the free space 125 is contiguous.

Furthermore, in the configuration shown several relevant structural parameters can be derived. By way of example but not by way of limitation, the volume fraction of the drug-containing fibers, φ_{f}, with respect to the volume of the dosage form 100 (or the volume of the drug-containing solid 101 or a representative control volume of the dosage form) may be estimated as:

$${φ}_{f} = \frac{π}{2} \frac{R}{λ} \qquad ( 1 a )$$

The specific surface area (area per unit volume of fibers 210), Aₛ, is approximately:

$${A}_{s} = \frac{2}{R} \qquad ( 1 b )$$

The length of fibers 120 per unit volume of the drug-containing solid, lᵥ, is roughly:

$${l}_{v} = \frac{{φ}_{f}}{{π R}^{2}} = \frac{1}{2 R λ} \qquad ( 1 c )$$

Also, the surface area of fibers 120 per unit volume of the drug-containing solid (or a representative control volume), Aᵥ, is about:

$${A}_{v} = \frac{2 {φ}_{f}}{R} = \frac{π}{λ} \qquad ( 1 d )$$

It will become obvious to a person of ordinary skill in the art after reading this specification carefully that φ_{f}, Aₛ, lᵥ, and Aᵥ, affect the disintegration rate and other relevant properties of a fibrous dosage form. Furthermore, it would be obvious to a person of ordinary skill in the art that Eqs. (1a)-(1d) must be adapted if the structure/arrangement/assembly (e.g. the three dimensional structural network) of fibers is changed.

FIG. 1c shows a dosage form 100 or drug-containing solid 101 with stacked, substantially parallel sheets 130 that are separated and spaced from adjoining sheets 130 by free spacings, λ_{f}. The free spacings define free spaces 135 that are substantially interconnected between said sheets 130, and thus are substantially interconnected through or across the dosage form 100 or drug-containing solid 101. The arrangement (or structural assembly) shown provides control of two structural variables essential for tailoring the properties of the drug-containing solid 101 or dosage form 100: the thickness of the sheets 130, h, (or the average thickness, h₀) and the spacing between the sheets 130 or segments thereof, λ (or alternatively the free spacing, λ_{f}). Unlike in the previous non-limiting examples shown in FIGS. 1a and 1b, however, the free space 135 may not be contiguous. Free spaces 135 may be separated (e.g., divided) by the sheets 130; they may be connected along the sheets 130 but not across the sheets 130.

Other non-limiting continuous structural assemblies of one or more repeatably arranged, extruded structural elements are presented in FIG. 2. FIG. 2a shows a dosage form 200 with unidirectionally aligned drug-containing fibers 210 that are (almost) closely packed. FIG. 2b is an example of a structure with interpenetrating fibers 220 and FIG. 2c shows a cross-ply arrangement of fibers with square cross section 230. FIG. 2d shows a continuous structural assembly of one or more sheets 240 and one or more fibers 241 or one or more beads 242. FIG. 2e presents a non-limiting structural assembly comprising one or more stacked sheets 250 having at least a perforation 253 (e.g., one or more perforations or at least a perforation in each layer of the stacked layers of sheets, etc.) so that interconnected free spaces 255 combine or unite across sheets 250.

More examples of how the elements may be structured, arranged, assembled, or stacked would be obvious to a person of ordinary skill in the art. All of them are within the spirit and scope of this invention.

### Compositions and Material Structures of Elements

The extruded structural elements typically comprise one or more active ingredients 117, 118, 127, 128, 137, 138, 218, 228, 238, 248, 258 (also referred to here as “drug”), and in most cases also one or more excipients 119, 129, 139, 219, 229, 239, 249, 259 (also referred to herein as “excipient”). If an element comprises at least one active ingredient and at least one excipient, the drug and excipient may be structured in the element in an ordered or “partially or completely disordered” manner. Moreover, by way of example but not by way of limitation, the structural features of the drug or the excipient in the element may, for example, comprise particles, beads, polygons, ellipsoids, cubes, tubes, rods, etc., or combinations thereof, and have a size at the nano-, micro-, meso-, or macro-scale.

More such examples of compositions and material structures of elements would be obvious to a person of ordinary skill in the art. All of them are within the scope of this invention.

### Drug Release From Elements

If the composition of an element consists of drug only, or if the drug is interconnected in the material structure of the element, the drug may be in direct contact with dissolution fluid upon immersion of the element in a dissolution fluid. Thus, in some embodiments, the drug may be released from an element by dissolution of drug into the dissolution fluid.

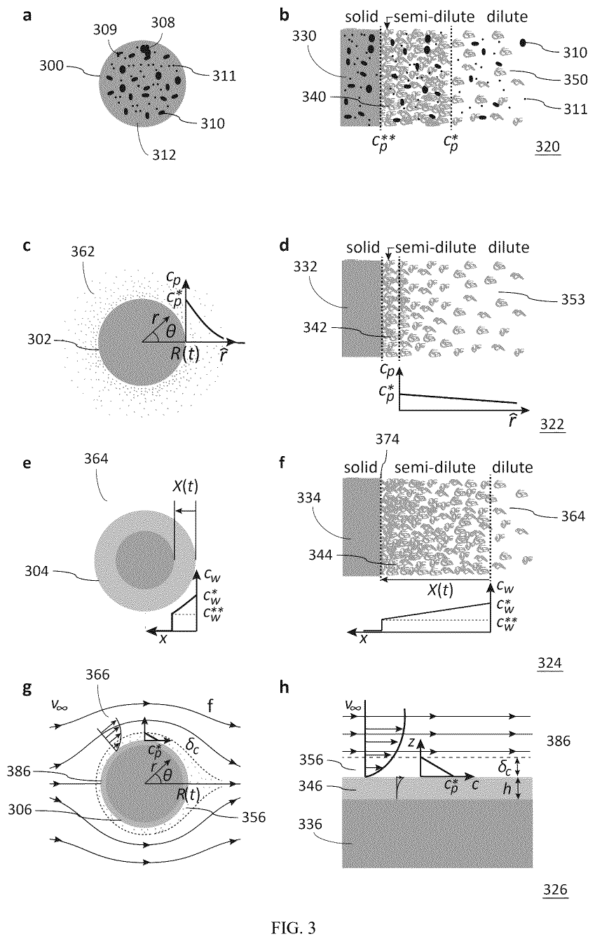

If the material structure of an element 300, however, comprises one or more discontinuous clusters of at least one drug particle 308, 310 or at least one drug molecule 309, 311 surrounded by a solid excipient 312 as shown in FIG. 3a erosion or swelling of the excipient 312 can be a prerequisite for drug release from the element 300. Two non-limiting examples of how drug may be released from such elements 300 are presented below.

In the first non-limiting example, the excipient comprises an erodible polymer. Thus, as soon as the element 300 is brought in contact with dissolution medium, the medium diffuses into the excipient. The penetrant molecules (e.g., the dissolution fluid that diffused into the solid excipient) may then induce the solid excipient to swell (e.g., to increase in volume) and to transition from a solid to a fluidic or gel consistency solution. Subsequently, the polymer molecules from the gel consistency solution may diffuse or erode into the dissolution medium. The drug may be released from the element 300 as soon as the excipient has converted to dissolved molecules or a gel with polymer concentration smaller than the “interfacial concentration”.

The “interfacial concentration” is referred to in this application as the polymer concentration which separates the “solid” and “liquid” regions. For a typical polymer that erodes into a dissolution fluid, the interface is diffuse, and thus the interfacial concentration is difficult to determine precisely. As schematically shown in FIG. 3b, the diffuse interface may extend over a layer 340 of non-negligible but finite thickness. It may be considered a semi-dilute gel consistency solution between the entangled, concentrated, and viscous polymer 330 (i.e., the “solid” or “semi-solid”) and the dilute, low-viscosity dissolution medium 350 (i.e., the “liquid”). Thus, typically, the concentration of an eroding polymer in the semi-dilute interfacial layer 340 (e.g., the “interfacial concentration”) is between the disentanglement concentration, cₚ*, of said polymer in a dissolution medium, and about the concentration, cₚ**, at which a solution comprising said polymer and a dissolution fluid becomes concentrated. (For further information, see e.g., P. G. De Gennes, “Scaling concepts in polymer physics”, fifth ed., Cornell University Press, 1996; or M. Doi, S. F. Edwards, “The theory of polymer dynamics”, Oxford University Press, 1986).

In the second non-limiting example, the excipient comprises an absorptive or swellable polymer. Thus upon immersion of the element in a dissolution fluid, the fluid diffuses into the solid polymeric excipient. The penetrant molecules (e.g., the dissolution fluid that diffused into the solid excipient) may then convert part or all of the solid drug enclosed in the polymeric excipient to dissolved drug molecules. The mobility of drug molecules may be greater in the penetrated polymeric excipient than in the excipient without penetrant. Thus the drug molecules embedded in the penetrated excipient may diffuse to the dissolution medium swiftly, and drug may be released within the specific time requirements.

More examples of drug release from elements would be obvious to a person of ordinary skill in the art. All of them are within the scope of this invention.

### Modeling Disintegration of Elements and Dosage Forms

The following examples set forth, in detail, ways by which the drug release and disintegration behavior of elements and structured dosage forms may be modeled. The models will enable one of skill in the art to more readily understand the properties and advantages of the structured dosage forms. The models and examples are presented by way of illustration, and are not meant to be limiting in any way.

### a) Element Erosion by Diffusion Without Convection

FIGS. 3c and 3d show a non-limiting example of a circular polymeric fiber (e.g., an element) 302 and its interface 322 after immersion in an unstirred, infinite dissolution medium 362. The polymer molecules are assumed to diffuse away from the interface faster than the dissolution medium diffuses into the fiber. Thus after a short wait after immersion, the thickness of the diffuse, semi-dilute layer 342 is (and remains) thin compared with the fiber radius or the thickness of the dilute region 352. The dissolution rate (or the disintegration rate) of the fiber 302 may thus be described by the diffusion of polymer molecules from the fiber interface into the dilute medium. The initial rate of erosion of the fiber 302 may be approximated by:

$$\frac{dR}{dt} = - \frac{{j}_{p}}{{ρ}_{e}} \approx - \frac{{c}_{p}^{*}}{{ρ}_{e}} \sqrt{\frac{{D}_{p}}{π t}} \qquad ( 2 )$$

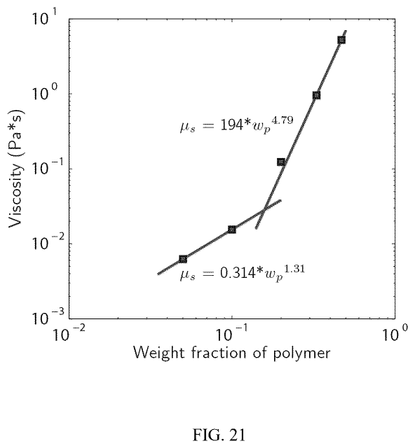

Integrating gives

$$R ( t ) = {R}_{0} - \frac{{c}_{p}^{*}}{{ρ}_{e}} \sqrt{\frac{4 {D}_{p} t}{π}} \qquad ( 3 )$$

where R(t) is the fiber radius as a function of time, R₀ is the initial fiber radius, jₚ the flux of the eroding polymer, ρₑ the density of the solid polymer, cₚ* the disentanglement concentration of the polymer (which is an estimate of the interfacial concentration and further described experimental examples 6 and 7, Eq. (18), and FIGS. 21, 22, and 23 later), and Dₚ the diffusivity of a polymer molecule in the dissolution medium.

By way of example but not by way of limitation, if R₀=250 μm, cₚ*=163 kg/m³, ρₑ=1150 kg/m³, Dₚ=1.09×10⁻¹⁰ m²/s, the fiber radius decreases to about 210 μm after the time t=R₀²/Dₚ=9.5 mins. Thus about 29% of the fiber are dissolved or disintegrated at this time in this example. By contrast, if the fiber radius is increased to 2.5 mm (a typical radius of a dosage form) and the other parameters are kept the same, only about 3% would be eroded 9.5 minutes after immersion in a still fluid. This percentage is an order of magnitude smaller than the corresponding value of a thin fiber, which exemplifies the advantage of a “thin” fiber over a “thick” fiber or dosage form for achieving fast disintegration (and high drug release) rates.

It would be obvious to a person of ordinary skill in the art that the model presented (and any of the following models) are readily adapted to fibers or elements of non-circular cross sections. Such fibers or elements include, but are not limited to fibers or elements with square, rectangular, elliptical, polygonal, or any other cross section. Furthermore, more examples of models of erosion of a single fiber or element in a still dissolution medium would be obvious to a person of ordinary skill in the art. All of them are within the scope of this invention.

### b) Diffusion of Dissolution Fluid Into an Element

FIGS. 3e and 3f present another non-limiting example of a circular polymeric fiber (e.g., an element) 304 and its interfacial region 324 after immersion in a dissolution fluid 364 that is of infinite extent and stagnant (not stirred). Now it is assumed that water (or dissolution fluid) diffusion into the polymer is faster than polymer diffusion into the fluid. This is opposite of the previous case. In this model, the thickness of the gel-layer 344 grows with time as dissolution fluid continues to diffuse in. Under Fickian diffusion (see, e.g., J. Crank, “The Mathematics of Diffusion”, second edition, Oxford University Press, 1975), the position of the solid/semi-dilute interface 374 is as follows, neglecting any form of erosion of the gelated layer:

$$X = {k}_{d} {t}^{\frac{1}{2}} \qquad ( 4 )$$

where t is time and k_{d} a constant.

If a substantial amount of dissolution fluid diffuses into the fiber 304, it swells and the polymer density (or the polymer concentration) in the fiber is reduced. The radius of the swollen, gelated fiber, R_{gel}, may be estimated as

$${R}_{g e l} = {{R}_{0} ( \frac{{ρ}_{e}}{{c}_{g e l}} )}^{\frac{1}{n}} \qquad ( 5 )$$

where R₀ is the initial fiber radius, the exponent n=3 for a fiber that expands uniformly in 3 dimensions (n=2 for a fiber that expands radially only), ρₑ is the density of the polymer in the solid/dry state, and c_{gel} an average concentration of swellable polymer in the gel 344.

The entire fiber 304 is converted into a gel when X=R_{gel}. Thus by Eq. (4), the time taken by the dissolution fluid 364 to penetrate the fiber 304 (i.e., to convert it into a gel) may be estimated as:

$${t}_{p e n} = \frac{{R}_{g e l}^{2}}{{k}_{d}} = \frac{{R}_{0}^{2}}{{D}_{eff}} \qquad ( 6 )$$

where D_{eff} is an effective diffusivity of physiological/body fluid in the polymeric fiber under physiological conditions. By way of example but not by way of limitation, if R₀=250 μm and D_{eff}=4×10⁻¹⁰ m²/s, by Eq. (6) tₚₑₙ=156 seconds. Conversely, if R₀ is increased to 2.5 mm and D_{eff} remains unchanged, tₚₑₙ increases to 260 minutes. Thus the penetration time of a “thin” fiber is much shorter than that of a “thick” fiber or a “thick” dosage form of the same composition.

It may be noted that the above equations can be readily adapted to multi-component fibers or elements. Also, more such examples of models of diffusion of dissolution fluid into a single fiber or element would be obvious to a person of ordinary skill in the art. All of them are within the scope of this invention.

### c) Disintegration of Penetrated Elements

The penetrated fiber or element may be considered a polymeric solution (or dispersion or gel) that has a viscosity greater than the viscosity of the dissolution fluid. If the viscosity of the solution (e.g., the penetrated fiber, or even the penetrated fiber surface) is small enough, and if such external forces applied on the fiber as gravity, shear, or imbalances in fluid pressure are large enough, the penetrated fiber may be deformed or broken up into pieces. The pieces may then dissolve or disentangle rapidly in the dissolution fluid. Thus a fiber may be disintegrated soon after it is penetrated in such non-limiting situations.

In other cases without limitation, a swollen, gelated (or penetrated) fiber may, for example, erode by diffusion of polymer molecules into a stagnant dissolution medium. This situation is similar to the non-limiting example shown in FIG. 3c and FIG. 3d. If the radius of the swollen, penetrated fiber is greater than the radius of the corresponding dry fiber, the swollen fiber has a greater surface area and a smaller polymer concentration (or density) than the dry fiber. Thus the swollen fiber disintegrates faster than the dry fiber in these non-limiting cases.

In both cases introduced above, the diffusion of dissolution fluid into the fiber or element contributes to faster disintegration. “Thin” fibers or elements are penetrated faster than “thick” fibers or elements, or “thick” minimally-porous dosage forms. “Thin” fibers or elements are therefore preferred to meet immediate-release specifications, the most relevant requirement of a typical pharmaceutical dosage form.

### d) Element Erosion With Convection

In a stirred medium, the moving dissolution fluid 366 may impose a shear stress on the surface of an element or fiber 386 (i.e., the surface of the gelated layer) and a concentration boundary layer 356 may develop around an element or fiber 306 as schematically shown in FIGS. 3g and 3h. Within the boundary layer, the concentration gradient may be substantial, but outside the layer it may be negligible. The concentration boundary layer thickness, dc, may decrease with increasing fluid velocity, or the Reynolds number. Hence the concentration gradient in the dissolution fluid 366 and thus also the material removal rate by convection of the eroding molecules away from the element or fiber surface 386 may increase.

The time to erode 80% of the content of a circular fiber or element of initial radius, R₀, in cross flow with Reynolds number, Re=2R₀ν_{∞}ρ_{f}/μ_{f}˜1 or smaller, may be estimated as:

$${t}_{E} ≅ 0.71 \times \frac{{ρ}_{e}}{{c}_{p}^{*}} \frac{{R}_{0}^{5 / 3}}{{D}_{p}^{2 / 3} {v}_{∞}^{1 / 3}} \qquad ( 7 )$$

where ν_{∞} is the far-field velocity of the dissolution medium, ρₑ the density of the eroding polymer in the fiber, cₚ* an estimate of the interfacial concentration, and Dₚ the diffusivity of a polymer molecule in the dissolution medium.

By way of example but not by way of limitation, if R₀=250 μm, cₚ*=163 kg/m³, ρₑ=1150 kg/m³, Dₚ=1.09×10⁻¹⁰ m²/s, and ν_{∞}=10 mm/s, by Eq. (7) t_{E}=1.7 mins. By contrast, if a fiber with initial radius R₀=2.5 mm would erode under the same conditions, the erosion time, t_{E}=77.8 min. Thus, also in this non-limiting example, the “thin” element or fiber disintegrates at least an order of magnitude faster than the “thick” fiber or the “thick” minimally-porous dosage form.

Any more examples of models of element or fiber erosion with convection would be obvious to a person of ordinary skill in the art. All of them are within the scope of this invention.

### e) Dosage Form Disintegration in a Stagnant Dissolution Fluid

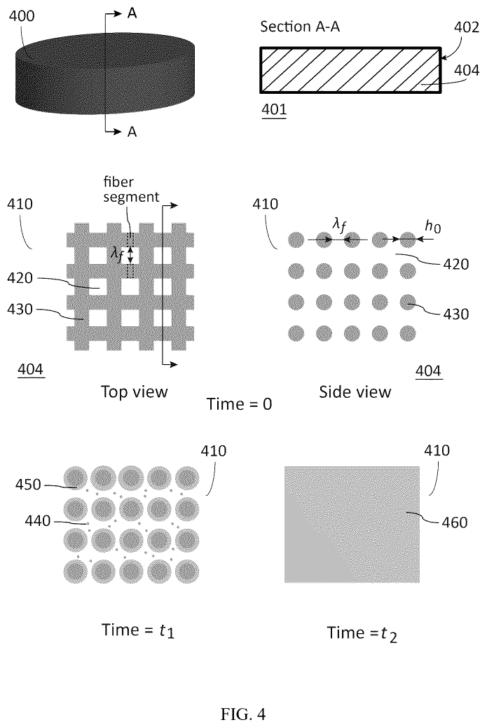

FIG. 4 presents a non-limiting example of the disintegration process of a structured dosage form 400 in a stagnant dissolution fluid 410. The structured dosage form 400 comprises a drug-containing solid 401 having an outer surface 402 and an internal structure 404 contiguous with and terminating at said outer surface 402. The internal structure 404 comprises a structural assembly of extruded structural elements or fibers 430. The extruded structural elements or fibers 430 contain an active ingredient and a polymeric excipient that is absorptive of or soluble in (e.g., erodible by) a dissolution medium 410. The elements 430 further comprise segments separated and spaced from adjoining segments by free spacings, λ_{f}, which define one or more substantially interconnected free spaces 420 in the drug-containing solid 401.

Upon immersion of the dosage form 400 in a dissolution fluid 410, the free spaces 420 may be percolated rapidly by the fluid 410 if (a) the free spaces 420 are (partially or entirely) inter-connected, (b) the content of the free spaces 420 is partially or entirely removable by the dissolution fluid 410, (c) the free spacing, λ_{f}, (e.g., the “free” distance between the one or more elements or segments) is on the sub-micro-, micro-, or meso-scale or greater, and (d) the excipient in the element or fiber is wettable by the dissolution fluid. Thus if the above conditions are satisfied, an element or fiber 430 in the three dimensional structural network will be surrounded by the dissolution fluid 410 soon (e.g. in less than about a second) after immersion of the dosage form 400. It is assumed that this is the case in the non-limiting example described here. The time to percolate part or all of the free spaces 420 is thus not considered to be rate-determining in dosage form disintegration or drug release.

Subsequent to fluid 410 percolation to the interior of the drug-containing solid 404, the dissolution fluid 410 that surrounds a segment then penetrates into it by diffusion, and the segment may swell and erode. Upon inter-diffusion of the fluid 410 and the polymeric segment, polymer molecules 440 (and gel-layer 450) may spread out. They may intersect with the molecules of adjoining segments at a certain time, t₁, after immersion. Then at t₂ a polymer-fluid solution 460 is formed. The time t₂ to convert the drug-containing solid 404 to such a solution 460 may be estimated by the penetration and erosion times of a single element (or a single segment) 430 in a stagnant fluid 410 (e.g. by Eqs. (3) and (6)).

If all the free spaces 420 are percolated by the dissolution fluid 410, and the drug containing solid 401 further does not expand as it is converted to a solution 460, the concentration of the excipient polymer, c_{p,sol}, in the solution 460 is about:

$${c}_{p , s o l} = \frac{{M}_{e}}{{V}_{e} + {V}_{fs}} = \frac{{φ}_{f} {φ}_{e} {ρ}_{e}}{1 - {φ}_{f} ( 1 - {φ}_{e} )} \qquad ( 8 )$$

where Mₑ is the mass and Vₑ the volume of the absorptive/soluble excipient, V_{fs} the volume of the free spaces 420, φ_{f} the volume fraction of the solid/dry elements in the dry dosage form, φₑ the volume fraction of the absorptive/soluble excipient polymer in the dry elements 430, and ρₑ is the density of the excipient in the dry state.

The solution 460 is dilute and the polymer molecules disentangled if the polymer concentration in the solution 460, c_{p,sol}≤cₚ*. This is the case if:

$${φ}_{f} \leq \frac{{c}_{p}^{*}}{( 1 - {φ}_{e} ) {c}_{p}^{*} + {φ}_{e} {ρ}_{e}} \qquad ( 9 )$$

Thus if Eq. (9) is satisfied, the polymer concentration in, or the viscosity of, the solution 460 is so small that the solution 460 is dilute or almost dilute. Consequently, the structured dosage form can be considered disintegrated as soon the single elements (or segments) 430 are eroded or penetrated. Dosage form 400 disintegration is determined solely by the behavior of a single element 430, and the inter-element interactions may be neglected. Thus for an element or fiber 430 geometry and properties of the composition as in the non-limiting examples a and b above, the dosage form 400 is disintegrated just a few minutes after immersion. This is well within immediate-release specification, which is one of the most relevant requirements of a typical pharmaceutical dosage form 400.

If the concentration of polymer in the solution 460, c_{p,sol}>>cₚ*, however, the solution 460 may be considered a viscous mass. The viscous mass (or the viscous solution, or the viscous dosage form) then erodes from its exterior surface by diffusion. The diffusion flux of the eroding polymer, jₚ, may be estimated as:

$${j}_{p} = \frac{{c}_{p}^{*} \sqrt{{D}_{p}}}{\sqrt{π t}} \qquad ( 10 )$$

and the time to disintegrate a thickness, H_{dis}, of the viscous mass 460 eroding from both faces may be estimated by

$${t}_{d i s} = \frac{{H}_{d i s}}{2} {{c}_{p , s o l} ( \frac{1}{{r}_{d i s}} {\int }_{0}^{{t}_{d i s}} {j}_{p} dt )}^{- 1} = \frac{π}{8} {( \frac{{c}_{p , sol}}{{c}_{p}^{*}} )}^{2} \frac{{H}_{d i s}^{2}}{{D}_{p}} \qquad ( 11 )$$

Thus by way of example but not by way of limitation, if c_{p,sol}=300 kg/m³, cₚ*=163 kg/m³, H_{dis}=1 mm, and Dₚ=1.09×10⁻¹⁰ m²/s, by Eq. (11), t_{dis}=203 min. This disintegration time does not meet immediate-release specifications, and is far longer than the time to penetrate or disintegrate a single element 430. Thus if the concentration of polymer in (and the viscosity of) the solution 460 are too high, the drug release rate of the fibrous dosage form may be reduced substantially. This is detrimental to an immediate-release dosage form.

It may be noted that in case the structural assembly of elements expands (and/or ruptures) after immersion in a dissolution medium, the relative amount of dissolution fluid in the solution 460 is increased. Thus the solution 460 is less concentrated and the threshold given by Eq. (9) can be increased.

Any more models or examples of the disintegration of a structured dosage form in a stagnant fluid obvious to a person of ordinary skill in the art are all within the scope of this invention.

### f) Dosage Form Disintegration in a Stirred Dissolution Fluid

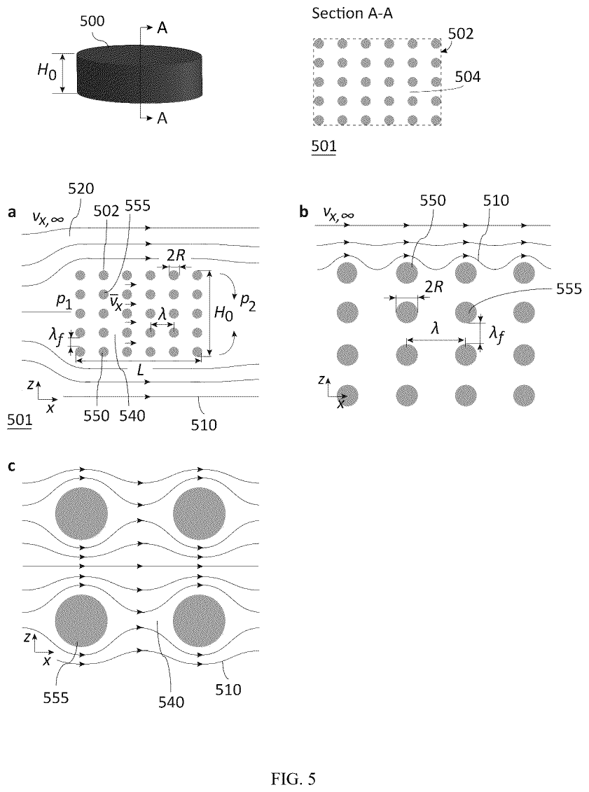

FIG. 5 presents a non-limiting example relevant to the disintegration of a fibrous dosage form (e.g., a non-limiting example of a structured dosage form) in a stirred dissolution medium. The fibrous dosage form 500 comprises a drug-containing solid 501 having an outer surface 502 and an internal structure 504 contiguous with and terminating at said outer surface 502. The outer surface 502 may comprise a solid, or a liquid, or a gas, and is defined as the plane spanned by the fibers 555 (or fiber segments) at the surface 502 of the drug-containing solid 501. The internal structure 504 comprises a structural assembly of extruded fibers 550, 555. The fibers 550, 555 contain an active ingredient and a polymeric excipient that is erodible by a dissolution medium 520. The fibers 550, 555 further comprise fiber segments separated and spaced from adjoining segments by free spacings, λ_{f}, which define one or more substantially interconnected free spaces 540 in the drug-containing solid 501.

FIG. 5a shows non-limiting examples of the streamlines 510 around the fibrous dosage form 500 in a stirred dissolution medium 520 with far-field velocity, ν_{x,∞}. The fluid velocity near the surface 502 is far greater than that in the interior 540. As a result, erosion of the fibers' surface planes is the greatest. If the inter-fiber spacing, λ, is much greater than the fiber diameter, 2R, the streamlines 510 bend around the fibers 550 and enter the space between them (FIG. 5b). They roughly follow the same paths as the ones near the surface of a single fiber in an infinite medium (FIG. 3g). Thus it may be assumed that the erosion rate of the exposed half (e.g., the “half fiber” of the exposed surface) equals that of a single fiber exposed to the same far-field velocity. For an initial fiber radius, R₀=250 μm, and a fluid velocity, ν_{x,∞}=10 mm/s, the erosion rate of a fiber on the dosage form surface for the parameter values given above may be derived from Eq. (7) as E=−dH/dt≈1087 nm/s. Accordingly, if surface erosion is from the two parallel faces of the dosage form 500, the time to erode 80 percent of a dosage form 500 that is 5 mm thick is: t_{dis}=0.8×H₀/2E≈38 min. This is, however, longer than what is desired for a typical immediate-release dosage form. (For further information on fluid flow and mass transfer around solid surfaces, see e.g., R. B. Bird, W. E. Stewart, E. N. Lightfoot, “Transport phenomena”, 2^{nd} edn., John Wiley & Sons, 2002, or L. Rosenhead, “Laminar boundary layers”, Oxford University Press, 1963).

Unlike the sequential layer-by-layer removal of material from the surface 502, material removal in the interior 540 of the dosage form is a parallel process because all the fibers 555 (e.g. the fibers in the interior) erode simultaneously. But the fibers 550, 555 impede fluid flow, reducing the fluid velocity in the interior of the structure (i.e., in the free spaces). The streamlines in the free spaces (or pores) may be as shown in FIG. 5c and an average fluid velocity in the free spaces, v̅ₓ, may be approximated by Darcy's law:

$${\overline{v}}_{x} = - \frac{1}{1 - {φ}_{f}} \frac{K}{{μ}_{l}} \frac{dp}{dx} \qquad ( 12 )$$

where μₗ is the viscosity of the liquid dissolution fluid, K is a hydraulic permeability and dp/dx a pressure gradient across the dosage form.

For a cross-ply arrangement of fibers as shown in FIG. 1b, where fibers of volume per unit length πR² are arranged in spaces of volume per length 2Rλ, the hydraulic permeability, K, in the x-direction may be estimated as

$$K = \frac{{K}_{⊥} + {K}_{\vert \vert}}{2} \qquad ( 13 a )$$

$$where$$

$${K}_{⊥} = \frac{R λ}{2 π} ( ln ( \sqrt{\frac{2 λ}{π R}} ) - \frac{4 - {{π}^{2} ( R / λ )}^{2}}{8 + 2 {{π}^{2} ( R / λ )}^{2}} ) \qquad ( 13 b )$$

$$and$$

$${K}_{\vert \vert} = \frac{R λ}{16} ( \frac{16}{π} ln ( \sqrt{\frac{2 λ}{π R}} ) + \frac{8 R}{λ} - \frac{π {R}^{2}}{{λ}^{2}} - \frac{12}{π} ) \qquad ( 13 c )$$

(for further information, see, e.g., J. Happel and H. Brenner, “Low Reynolds number hydrodynamics with special application to particulate media”, Prentice-Hall, Englewood Cliffs, NJ, 1965). Some estimated values of K, K_{⊥}, and K_{∥} are listed below for specific non-limiting examples of the radius of solid fibers, R, and the inter-fiber spacing, λ:

| | R | λ | K_{⊥} | K_{\|\|} | K |
|---|---|---|---|---|---|
| | (μm) | (μm) | (m²) | (m²) | (m²) |
| B | 245 | 1783 | 2.2 × 10⁻⁸ | 3.1 × 10⁻⁸ | 2.7 × 10⁻⁸ |
| C | 253 | 922 | 2.9 × 10⁻⁹ | 4.1 × 10⁻⁹ | 3.5 × 10⁻⁹ |
| D | 243 | 629 | 4.6 × 10⁻¹⁰ | 7.1 × 10⁻¹⁰ | 5.9 × 10⁻¹⁰ |

The pressure gradient across the dosage form 500 may be estimated from fluid flow outside the dosage form 500 (FIG. 5a). Far away from the dosage form 500, the dissolution fluid 520 is inviscid, at ambient pressure, and flowing towards the dosage form 500 at a velocity ν_{x,∞}. Near the front of the dosage form, however, the flow bifurcates, the streamlines 510 divide, and the fluid pressure increases. The relation between fluid pressure, p, and fluid velocity, νₗ, in the free-flowing medium (outside the dosage form) may be described by Bernoulli's equation as p=pₐₜₘ+0.5ρₗ(ν_{x,∞}²-νₗ²) where ρₗ is the density of the liquid medium. Thus if it is assumed that νₗ≈0 at the front of the dosage form, the pressure at the front of the dosage form, p₁, is about p₁≈pₐₜₘ+0.5ρₗν_{x,∞}².

Further assuming that p≈pₐₜₘ at the rear of the dosage form, the pressure gradient may be estimated as:

$$\frac{dp}{dx} ≅ \frac{Δ p}{L} ≅ \frac{1}{2} \frac{{ρ}_{l} {v}_{x , ∞}^{2}}{L} \qquad ( 14 )$$

where a cord length L≈D/² may be used for a dosage form that is of cylindrical disk shape (D is the dosage form diameter). Thus the average velocity of the fluid in the free spaces (or pores), v̅ₓ, may be estimated by combining Eqs. (12)-(14).

If the pores are considered an array of tubes, the maximum fluid velocity in the pores (e.g., the free spaces) is a factor two greater than the average velocity, v̅ₓ. Here we insert the maximum velocity as the fluid velocity, ν_{∞}, in Eq. (7) to calculate the erosion time of the fibers 555 in the interior of the structural assembly. The following estimated velocities and erosion times, t_{E}, are obtained for the conditions under which the non-limiting experimental examples (shown later and summarized in Table 1) were performed:

| | R₀ | λ₀ | v̅ₓ | v_{∞} | t_{E} | t_{0.8} |
|---|---|---|---|---|---|---|
| | (μm) | (μm) | (μm/s) | (μm/s) | (min) | (min) |
| B | 245 | 1783 | 346 | 692 | 4.5 | 5 .64 |
| C | 253 | 922 | 61 | 122 | 8 | 9.14 |
| D | 243 | 629 | 15 | 30 | 12 | 14.17 |

(Here again the calculations refer to a structure/arrangement/assembly as shown in FIG. 1b. The parameter values cₚ*=163 kg/m³, ρₑ=1150 kg/m³, Dₚ=1.09×10⁻¹⁰ m²/s, ρₗ=1000 kg/m³, μₗ=0.001 Pa·s, ν_{x,∞}=10 mm/s, and L=10 mm are used in combination with Eqns. (7) and (12)-(14). The values of the hydraulic conductivity, K, were assumed time-invariant in the calculations and are based on the initial radius, R₀, and the initial inter-fiber distance, λ₀. t_{0.8} is the measured time to dissolve 80 percent of the drug content from the experimental dosage forms.)

The calculated t_{E} values are well within immediate-release specification, and shorter than the times to disintegrate the dosage form structures from the exterior surfaces. Thus even though the velocity in the interior of the fibrous structure 504 is reduced substantially, material removal by simultaneous erosion of fibers 555 in the interior is faster than by sequential erosion from the surface in the non-limiting examples presented.

It may be noted, however, that even in a stirred medium, if swelling of fibers in the interior is faster than erosion, the fibrous dosage form may disintegrate as described in the non-limiting example e above.

Finally, for a non-porous disk-shaped solid dosage form that erodes from both faces by convection (e.g., in a rotating basket of a USP dissolution apparatus), the erosion rate per eroding face may be approximated as:

$$E = - \frac{dH}{dt} = 0.62 ( \frac{{D}_{p} {c}_{p}^{*}}{{ρ}_{e}} ) {( \frac{{μ}_{l}}{{D}_{p} {ρ}_{l}} )}^{\frac{1}{3}} {( \frac{{ρ}_{l} Ω}{{μ}_{l}} )}^{\frac{1}{2}} \qquad ( 15 )$$

where Ω is the angular velocity of the rotating basket. The effective disintegration time of the dosage form of initial thickness H₀ eroding from both faces may be estimated as:

$${t}_{dis} = \frac{{H}_{0}}{2} \frac{1}{dH / dt} \qquad ( 16 )$$

(It may be noted that in the present non-limiting example, erosion from the sides is not considered because the thickness of the dosage form is assumed smaller than the dosage form width or length. Furthermore, we may note that the model may be adapted if the eroding surfaces are not planar.)

By way of example but not by way of limitation, if cₚ*=163 kg/m³, Dₚ=1.09×10⁻¹⁰ m²/s, ρₑ=1150 kg/m³, ρₗ=1000 kg/m³, μₗ=0.001 Pa·s, Ω=5.24 rad/s, and H₀=5 mm, by Eqs. (15) and (16) the calculated 0.8×t_{dis}=73 min. This estimation of the disintegration time is an order of magnitude greater than the values tabulated above for parallel erosion of fibers with flow through the fibrous structure. Thus also in a stirred medium, the fibrous structures are superior to the non-porous structures if immediate drug release is the goal.

(For further details related to the USP dissolution apparatus, see, e.g., The United States Pharmacopeial Convention, USP 39-NF 34; further details related to convective mass transfer models are given, e.g., in V. G. Levich, “Physicochemical Hydrodynamics”, Prentice-Hall, Englewood Cliffs, NJ, 1962.)

Any more models or examples of the disintegration of a fibrous or structured dosage form in a stirred fluid obvious to a person of ordinary skill in the art are all within the scope and spirit of this invention.

### g) Summary of Disintegration Models

The above non-limiting models illustrate the effects of the following design parameters on the disintegration rate of elements and structured dosage forms: the geometry of the structural assembly of elements, the solubility of the excipient in the dissolution medium (e.g., the “interfacial concentration”), the diffusivity of the excipient in the dissolution medium, the diffusivity of the medium in the excipient, the fractions of the individual components in the elements, and the disentanglement concentration of the excipient. All these parameters can be deterministically controlled during the manufacture of a structured dosage form.

Furthermore, the models illustrate that the structured dosage forms can be so designed that the length-scale of the disintegration-rate-determining mass transfer step is decreased from the thickness of the dosage form to the half-thickness of an element. As a result, the structured dosage forms can be designed to deliver drug faster (e.g., an order of magnitude faster) than the corresponding non-porous solid forms. Thus, the structured dosage forms offer predictable disintegration within a wide range of disintegration (and drug release) rates.

### Dosage Form Design Features

In view of the theoretical models and considerations above, which are suggestive and approximate rather than exact, the design and embodiments of the structured dosage forms disclosed herein comprise the following.

The pharmaceutical dosage forms disclosed herein generally comprise a drug-containing solid having an outer surface and an internal structure contiguous with and terminating at said outer surface. The internal structure comprises a structural assembly of one or more extruded structural elements. The elements comprise at least one active ingredient, and in some cases also at least one excipient. The elements further comprise segments separated and spaced from adjoining segments by free spacings defining one or more substantially interconnected free spaces in the drug-containing solid.

For achieving rapid percolation of dissolution fluid into the free spaces, in some embodiments a “free spacing”, λ_{f}, (e.g., a “free” distance between adjoining (i.e., neighboring) elements, or adjoining segments, is such that the percolation time of physiological/body fluid into one or more interconnected free spaces of the dosage form is no greater than 900 seconds under physiological conditions. This includes, but is not limited to percolation times no greater than 700 seconds, no greater than 500 seconds, no greater than 300 seconds, no greater than 100 seconds, no greater than 50 seconds, or no greater than 10 seconds under physiological conditions. The pressure of the physiological/body fluid at different surfaces of the interconnected free spaces may assume different values during fluid percolation.

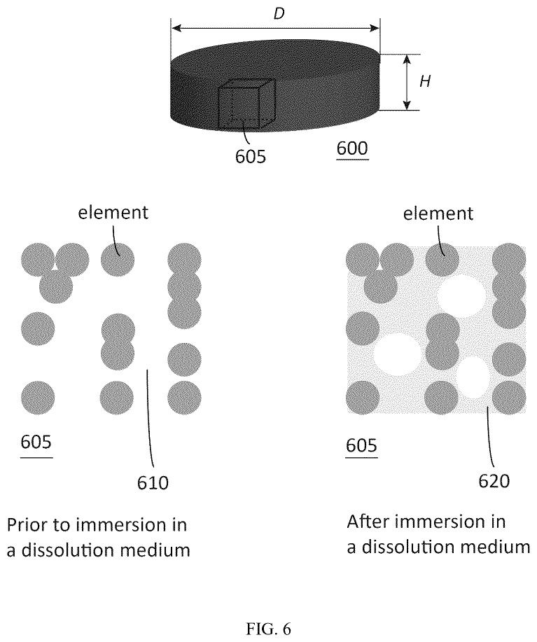

By way of example but not by way of limitation, the percolation time into one or more interconnected free spaces of the dosage form may be determined as follows (FIG. 6). First a volume 605 of the dosage form 600 may be identified that contains one or more interconnected free spaces 610. Then the volume of the interconnected free spaces 610 in said volume of the dosage form 605 may be determined. Then said volume of the dosage form 605 may be immersed in a dissolution medium. Then the volume of dissolution medium 620 that percolated into the volume of the interconnected free spaces 610 of said volume of the dosage form 605 may be determined. As soon as the volume of dissolution medium 620 that percolated into the volume of the interconnected free spaces 610 of said volume of the dosage form 605 is greater than 20 percent of the initial volume of the interconnected free spaces 610, the volume of the interconnected free spaces 610 of said volume of the dosage form 605 may be considered percolated.

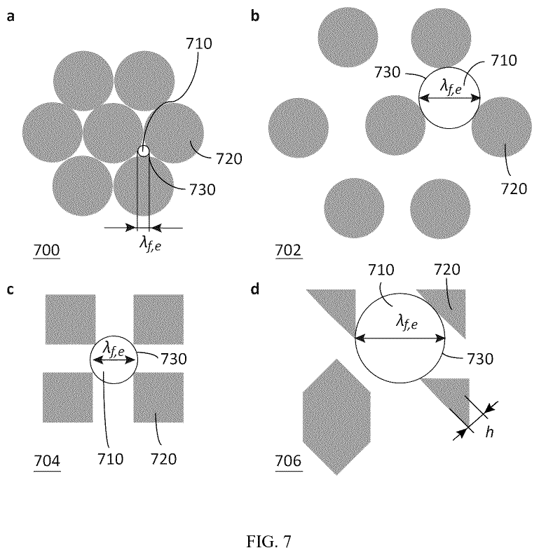

Also, in some embodiments, the effective free spacing, λ_{f,e}, on average is greater than 0.1 μm. This includes, but is not limited to an average λ_{f,e} greater than 0.25 μm, or greater than 0.5 μm, or greater than 1 μm, or greater than 2 μm, or greater than 5 μm, or greater than 7 μm, or greater than 10 μm, or greater than 15 μm, or greater than 20 μm, or greater than 25 μm, or greater than 30 μm, or greater than 40 μm, or greater than 50 μm, or in the ranges of 0.1 μm-5 mm, 0.1 μm-3 mm, 0.25 μm-5 mm, 0.5 μm-5 mm, 0.25 μm-3 mm, 0.1 μm-2.5 mm, 0.25 μm-2 mm, 1 μm-4 mm, 5 μm-4 mm, 10 μm-4 mm, 15 μm-4 mm, 20 μm-4 mm, 30 μm-4 mm, 40 μm-4 mm, or 50 μm-4 mm. As shown in the non-limiting 2-D examples 700, 702, 704, 706 of FIG. 7, the “effective free spacing” between adjoining segments is defined as the maximum diameter of a sphere that fits in the corresponding free space 710 considering the elements 720 as rigid, fixed bodies. The diameter of such spheres may be estimated from 2-d images of the microstructure. Such 2-d images may be obtained from scanning electron micrographs of the cross section of the dosage form. The greatest circles 730 that fit in the free spaces 710 of the microstructure may be drawn on the scanning electron micrograph (e.g., the 2-d image) and the area-based average diameter of the circles 730 (e.g., the average effective free spacing) calculated.

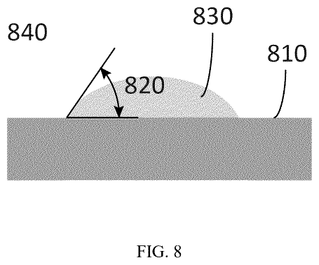

Furthermore, in some embodiments at least one of the one or more excipients is wettable by a physiological/body fluid under physiological conditions. In the context of this work, a solid surface 810 is wettable by a fluid if the contact angle 820 of a fluid droplet 830 on the solid surface 810 exposed to air 840 is no more than 90 degrees (FIG. 8). In some embodiments, the contact angle may not be stationary. In this case, in the invention herein a solid surface is wettable by a fluid if the contact angle 820 of a fluid droplet 830 on the solid surface 810 exposed to air 840 is no more than 90 degrees at least 60-360 seconds after the droplet 830 has been deposited on the surface.

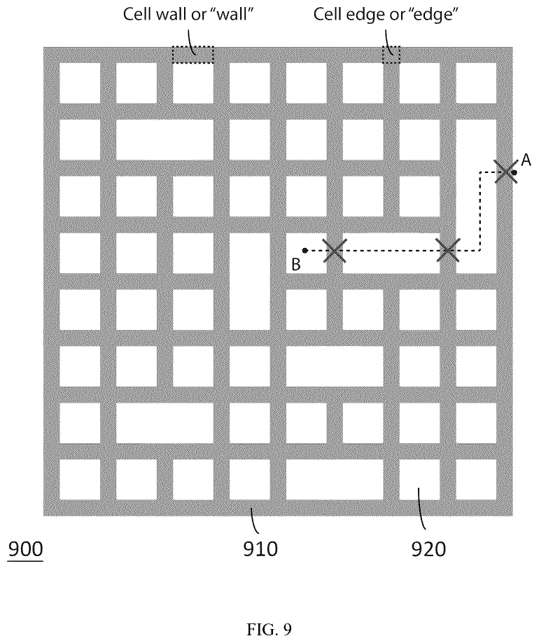

In some embodiments disclosed herein, moreover, the following holds. If the average wall thickness is greater than 100 μm, an interconnected, continuous cluster of free space that extends from the outer surface of the drug-containing solid to a given point in the internal structure is obtained if no more than 0 to 12 walls are ruptured (e.g, walls of drug-containing solid enclosing free space are opened or removed). This includes, but is not limited to 0-11, 0-10, 0-9, 0-8, 0-7, 0-6, or 0-5 walls that must be ruptured to obtain an interconnected cluster of free space that extends from the outer surface to a given point in the internal structure. If the average wall thickness is smaller than 100 μm, no more than 0 to 24 walls must be ruptured to obtain such an interconnected cluster of free space. This includes, but is not limited to 0-22, 0-22, 0-18, 0-16, 0-14, 0-12, or 0-10 walls that must be ruptured to obtain an interconnected cluster of free space that extends from the outer surface of the drug-containing solid to a given point in the interior. In FIG. 9, a 2-d example without limitation 900 is presented that shows 3 walls 910 to be ruptured for obtaining an interconnected cluster of free space 920 from point A to point B.

For achieving a specific surface area (i.e., surface area-to-volume ratio) large enough to guarantee rapid disintegration of the elements, in some embodiments the one or more elements have an average thickness, h₀, no greater than 2.5 mm. This includes, but is not limited to h₀ no greater than 2 mm, or no greater than 1.5 mm. It may be noted, however, that if the elements are very thin and tightly packed, the free spacing between the elements can be very small, too. This may limit the rate at which dissolution fluid can percolate or flow through the internal structure upon immersion in a dissolution fluid. Thus, in some embodiments the one or more elements have an average thickness, h₀, in the ranges of 0.1 μm-2.5 mm, 0.5 μm-2.5 mm, 1 μm-2.5 mm, 1.75 μm-2.5 mm, 2.5 μm-2.5 mm, 2.5 μm-2 mm, 5 μm-2 mm, 10 μm-2 mm, 15 μm-2.5 mm, 20 μm-2.5 mm, 30 μm-2.5 mm, 40 μm-2.5 mm, or 50 μm-2.5 mm. We may further note that the average thickness of the elements, h₀, can be greater than 2.5 mm in dosage forms that release drug over longer periods of time (e.g., in a time greater than about 25-45 minutes).

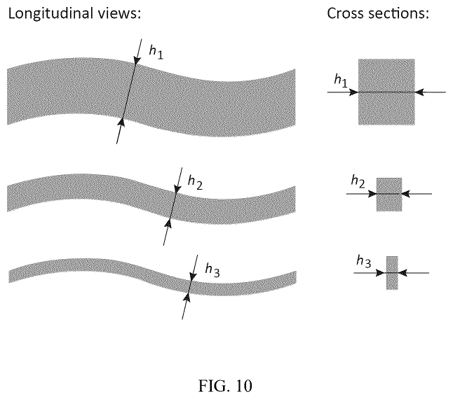

The element thickness, h, may be considered the smallest dimension of an element (i.e., h≤w and h≤l, where h, w and l are the thickness, width and length of the element, respectively). The average element thickness, h₀, is the average of the element thickness along the length of the one or more elements. By way of example but not by way of limitation, FIG. 10 presents three elements of equal length but different thicknesses. In this non-limiting example, the average element thickness, h₀=(h₁+h₂+h₃)/3. Both the average element thickness, h₀, and the thickness of a specific element at a specific position, h, may, for example, be derived from scanning electron micrographs of the cross section of the dosage form.

Furthermore, in some embodiments, a contact width, 2a, between two elements (or two segments) is no greater than 2.5 mm. This includes, but is not limited to a contact width between two elements (or two segments) no greater than 2 mm, or no greater than 1.75 mm, or no greater than 1.5 mm. In other examples without limitation, a contact width, 2a, between two elements (or two segments) may be no greater than 1.1 times the thickness of the contacting elements (or segments) at the position of the contact. This includes, but is not limited to a contact width, 2a, between two elements (or two segments) no greater than 1 time, or no greater 0.8 times, or no greater than 0.6 times the thickness of the contacting elements (or segments) at the position of the contact.

Moreover, we may note that the cross section of an element may, for example, be polygonal, ellipsoidal, etc. (or combinations thereof), and it may comprise inward-curved or outward-curved or un-curved surfaces. Furthermore, the cross section of an element may vary along the length (and/or along the width) of the element.

An element or a segment in the structural assembly may, for example, be defined by its position (e.g., the position of its central axis, etc.) relative to a reference point or frame. (In the invention herein, a reference frame may be understood as a reference coordinate system.) The reference point or the origin and orientation of the reference frame may be specified on the outer surface or within the internal structure of the drug containing solid.

In some embodiments of the invention herein, the position of at least one element or at least one segment in the structural assembly of one or more elements is precisely controlled. Such embodiments include, but are not limited to structural assemblies of elements wherein the position of a fraction of the elements or segments is precisely controlled. The volume fraction of elements or segments (with respect to the total volume of elements or segments that make up the structural assembly of elements) of which the position is precisely controlled can be greater than 0.1, or greater than 0.3, or greater than 0.5, or greater than 0.7, or greater than 0.9. It may be noted that in the context of the invention herein, a variable or a parameter (e.g., the position of an element, or a spacing between elements or segments, or an element thickness) is precisely controlled if it is deterministic and not stochastic (or random). A variable or parameter may be deterministic if, upon multiple repetitions of a step that includes said variable, the standard deviation of the values of said variable is smaller than the average value. This includes, but is not limited to a standard deviation of the values of said variable smaller than half the average value, or smaller than one third of the average value, or smaller than a quarter of the average value, or smaller than one fifth or the average value, or smaller than one sixth of the average value of said variable, or smaller than one eight of the average value of said variable, or smaller than one tenth of the average value of said variable, or smaller than one fifteenth of the average value of said variable, or smaller than one twentieth of the average value of said variable.

In some embodiments, furthermore, at least one spacing between elements or segments, λ, and/or at least one element thickness, h, is/are precisely (or deterministically) controlled. Thus, in some embodiments herein, if an element is produced multiple times under identical conditions, the standard deviation of the thickness of said elements is less than the average value of said elements' thickness. Similarly, if a spacing between elements is produced multiple times under identical conditions, the standard deviation of said inter-element spacing is less than the average value of said inter-element spacing in certain embodiments of the invention herein. It may be noted that in the invention herein, an inter-element spacing includes, but is not limited to a spacing between two segments. Also, we may note that the spacing between elements or segments may change along the length of said elements or segments. Similarly, an element thickness includes, but is not limited to the thickness of a segment. Also, the thickness of an element or a segment may change along the length of said element or segment.

A non-limiting example of a structural assembly of elements wherein the position of a large fraction (or all) of the elements, the spacing between elements, and the element thickness are controlled (or precisely controlled) is an ordered structure. As shown in the non-limiting schematics of FIGS. 1 and 2, such regular or ordered structures can, for example, comprise multiple layers of fibers or fiber segments that are stacked. The fibers or fiber segments in a layer can be oriented parallel (or almost parallel) to each other in some non-limiting ordered structures herein. The advantage of ordered structures over disordered or random structures is that the properties, such as the drug release rate by the structure, can be better controlled.

Moreover, as shown in FIGS. 1 and 2, in some embodiments herein the structural assembly of one or more elements may comprise inter-element contacts (e.g., contacts between elements and/or segments). Such inter-element contacts include, but are not limited to point contacts (as schematically shown in FIGS. 1a and 1b) or line contacts as schematized in FIGS. 2a and 2d (for further information related to point contacts and line contacts, see, e.g., K. L. Johnson, “Contact mechanics”, Cambridge University Press, 1985). The inter-element contacts may provide mechanical support to the structure (e.g., the structural assembly of elements). They may, however, also hold up disintegration and dissolution of the structure upon immersion in a dissolution medium. Thus, in some embodiments the number of inter-element contacts in a structured dosage form or drug-containing solid, and/or at least one position of an inter-element contact in a structured dosage form, and/or a contact width of at least one inter-element contact in a structured dosage form is/are precisely controlled in the structural assembly of one or more elements. This includes, but is not limited to embodiments wherein the position of a fraction of the inter-element contacts is precisely controlled, said fraction being greater than 0.3 or greater than 0.5. This further includes, but is not limited to embodiments wherein the contact width of a fraction of the inter-element contacts is precisely controlled, said fraction being greater than 0.3 or greater than 0.5.

Typically, the volume fraction of drug-containing elements in the dosage form is no greater than 0.98. In other non-limiting examples, the volume fraction of drug-containing elements in the dosage form is no greater than 0.95, no greater than 0.93, or no greater than 0.9. In most cases, it is in the range 0.1-0.9, depending on how the one or more elements are arranged. A small volume fraction of drug containing elements is desirable to fill small amounts of drug in a comparable large volume (i.e., if the dosage form is used for delivery of a highly potent drug with a drug dose of just a few milligrams or less). On the contrary, a large volume fraction of drug-containing elements is desirable to fill large amounts of drug in a small volume (i.e., if the dosage form is used for delivery of a low potency drug or delivery of multiple active ingredients with a total drug dose of several 100 mg or more).

For achieving rapid erosion of one or more elements after contact with physiological/body fluid, in some embodiments the drug-containing elements include at least one excipient that has a solubility greater than 0.1 g/l in physiological/body fluids under physiological conditions. This includes, but is not limited to a solubility of at least one excipient in a physiological/body fluid greater than 0.5 g/l, or greater than 1 g/l, or greater than 5 g/l, or greater than 10 g/l, or greater than 20 g/l, or greater than 30 g/l, or greater than 50 g/l, or greater than 70 g/l, or greater than 100 g/l. Furthermore, the diffusivity of a dissolved excipient molecule in a physiological/body fluid may be greater than 1×10⁻¹² m²/s under physiological conditions. This includes, but is not limited to a diffusivity of a dissolved excipient molecule in a physiological/body fluid greater than 2×10⁻¹² m²/s, greater than 4×10⁻¹² m²/s, greater than 6×10⁻¹² m²/s, greater than 8×10⁻¹² m²/s, or greater than 1×10⁻¹¹ m²/s under physiological conditions. The volume fraction of soluble excipient in the excipient (e.g., the excipient in its totality or all the volume of the one or more excipients in the one or more fibers) may be greater than 0.02. This includes, but is not limited to volume fractions of the soluble excipient in the excipient greater than 0.04, greater than 0.06, greater than 0.08, or greater than 0.1.

In polymers that form viscous solutions when combined with a dissolution medium, the ‘solubility’ in the context of this invention is the polymer concentration in physiological/body fluid at which the average shear viscosity of the polymer-physiological/body fluid solution is 5 Pa·s in the shear rate range 1-100 l/s under physiological conditions. The pH value of the physiological/body fluid may thereby be adjusted to the specific physiological condition of interest. By contrast, the solubility of a material that does not form a viscous solution when combined with a dissolution medium is the maximum amount of said material dissolved in a given volume of dissolution medium at equilibrium divided by said volume of the medium. It may, for example, be determined by optical methods.

Furthermore, in some embodiments the one or more drug-containing elements include at least one excipient that is absorptive of a physiological/body fluid. The effective diffusivity of physiological/body fluid in an absorptive excipient (and/or an element) is greater than 0.5×10⁻¹¹ m²/s under physiological conditions. In other examples without limitation, the effective diffusivity of physiological/body fluid in an absorptive excipient (and/or an element) may be greater than 1×10⁻¹¹ m²/s, greater than 3×10⁻¹¹ m²/s, greater than 6×10⁻¹¹ m²/s, or greater than 8×10⁻¹¹ m²/s under physiological conditions.

Alternatively, for absorptive excipients where diffusion of physiological/body fluid to the interior is not Fickian, a rate of penetration may be specified. In some embodiments, the rate of penetration of a physiological/body fluid into a solid, absorptive excipient (and/or an element) is greater than an average thickness of the one or more drug-containing elements divided by 3600 seconds (i.e., h₀/3600 μm/s). In other examples without limitation, rate of penetration may be greater than h₀/1800 μm/s, greater than h₀/1200 μm/s, greater than h₀/800 μm/s, or greater than h₀/600 μm/s.

For determining the effective diffusivity (and/or the rate of penetration) of dissolution medium in a solid, absorptive excipient (and/or an element) the following procedure may be applied. An element (e.g an element of the dosage form structure or an element that just consists of the absorptive excipient) may be fixed at both ends and placed in a still dissolution medium at 37° C. The time t₁ for the element to break apart or deform substantially may be recorded. (By way of example but not by way of limitation, a deformation of a fiber may be considered substantial if either the length, width, or thickness of the fiber differs by more than 10 to 20 percent from its initial value. In elements with weight fraction, wₑ, or volume fraction, φₑ, of absorptive/swellable excipient smaller than 0.4, a deformation of an element may be considered substantial if either the length, width, or thickness of the element differs by more than 25×φₑ percent or 25×wₑ percent from its initial value.) The effective diffusivity, D_{eff}, may then be determined according to D_{eff}=h_{f}²/4t₁ where h_{f} is the initial element thickness (e.g., the thickness of the dry element). Similarly, the rate of penetration of a physiological/body fluid into the element is equal to h_{f}/2t₁.

The effective diffusivity of dissolution medium in or the average velocity at which the fluid front advances (i.e., the rate of penetration of a physiological/body fluid) into a solid, absorptive excipient (or an element) may also be determined by spectral methods. By way of example but not by way of limitation, a film with thickness of the order of the thickness of an element may be cast from the element material (or the absorptive excipient only) by either addition and removal of a solvent or by melting and solidification. One side of the film may be exposed to the dissolution medium. On the other side of the film, the concentration of dissolution medium may be monitored. As soon as the monitored concentration of dissolution medium raises substantially (e.g., as soon as the concentration of water or dissolution fluid in the absorptive/swellable excipient on the monitored surface is greater than twice the concentration of water or dissolution fluid in the absorptive/swellable excipient of the initial solid film or element), the film is penetrated. The time/1 to penetrate the film may be recorded and the effective diffusivity and rate of penetration calculated as detailed in the previous paragraph. Spectral methods are suited for materials that have some mechanical strength (i.e., increased viscosity) when they are penetrated by the dissolution fluid. They are also suited for materials (or fibers) where the deformation of the fiber upon penetration of dissolution fluid is small.

In some embodiments, at least one excipient of the drug-containing solid transitions from solid to a fluidic or gel consistency solution upon being solvated with a volume of physiological/body fluid equal to the volume of the one or more free spaces of the drug-containing solid (or dosage form). To ensure that the disintegration rate of such a drug-containing solid is of the order of the disintegration rate of a single element (e.g., to avoid that the drug-containing solid forms a viscous mass upon immersion in a dissolution medium that erodes slowly from its outer surfaces), the viscosity of said solution is no greater than 500 Pa·s. In other words, a solution comprising the weight of soluble/absorptive excipient in the drug-containing containing solid and a volume of physiological/body fluid equal to the volume of the free spaces of the drug-containing solid (specifically the volume of the free spaces that are removable by the dissolution fluid), has a viscosity no greater than 500 Pa·s. This includes, but is not limited to a viscosity of said solution less than 400 Pa·s, less than 300 Pa·s, less than 200 Pa·s, less than 100 Pa·s, less than 50 Pa·s, less than 25 Pa·s, or less than 10 Pa·s. In the context of this work, the viscosity of a solution is the average shear viscosity of the solution in the shear rate range 1-100 l/s under physiological conditions.

Non-limiting examples of excipients that if used at the right quantities satisfy some or all of the above requirements include polyethylene glycol (PEG), polyvinylpyrrolidone (PVP), PEG-PVP copolymer, poloxamer, lauroyl macrogol-32 glycerides, polyvinylalcohol (PVA), PEG-PVA copolymer, polylactic acid, polyvinylacetate phthalate, polymethacrylates (e.g., poly (methacrylic acid, ethyl acrylate) 1:1, butylmethacrylat-(2-dimethylaminoethyl) methacrylat-methylmathacrylat-copolymer), gelatin, cellulose or cellulose derivatives (e.g., microcrystalline cellulose, hydroxypropyl cellulose, hydroxyethyl cellulose, methyl cellulose, hydroxypropyl methyl ether cellulose, hydroxypropyl methylcellulose), starch, polylactide-co-glycolide, polyvinyl caprolactam-polyvinyl acetate-polyethylene glycol graft copolymer, pregelatinized starch, lactose, sodium starch glycolate, polyacrylic acid, acrylic acid crosslinked with allyl sucrose or allyl pentaerythritol (e.g., carbopol), or polyols (e.g., lactitol, maltitol, mannitol, isomalt, xylitol, sorbitol, maltodextrin, etc.), among others.

The one or more free spaces may be filled with a matter selected from the group comprising solid, liquid, gas (or vacuum), or combinations thereof. If one or more elements (or one or more segments) is/are partially or entirely surrounded by free space, the content of said free space may be removed partially or entirely after contact with dissolution fluid to give the fluid access to the one or more elements within the structural assembly of elements. This condition is, for example, satisfied by gases. Examples of biocompatible gases that may fill the free space include air, nitrogen, CO2, argon, oxygen, and nitric oxide, among others.

Liquids that are partially or entirely removed from the structure upon contact with dissolution fluid, and thus may be used to fill the free spaces include, but are not limited to such biocompatible low viscosity fluids as: Polyethylene glycol (PEG) with molecular weight smaller than about 1000 Da (e.g. PEG 400, PEG 300, etc.), Poloxamer 124, 2-Pyrrolidone, Glycerol triacetate (Triacetin), D-alpha tocopheryl polyethylene glycol 1000 succinate (TPGS), Polyoxyl Hydroxystearate, Polyoxyl 15 Hydroxystearate, Castor oil, Polyoxyl castor oil (Polyethoxylated castor oil), Polyoxyl 35 castor oil, Polyoxyl hydrogenated castor oil, Glyceryl monooeleate, Glycerin, Propylene glycol, Propylene carbonate, Propionic acid, Peanut oil, water, Sesame oil, Olive oil, Almond oil, combinations of such (and/or other) liquids with a polymer or any other molecule that dissolves in them, among others.

Non-limiting examples of solids that are removed or dissolved after contact with physiological/body fluid include sugars or polyols, such as Sucrose, Lactose, Maltose, Glucose, Maltodextrin, Mannitol, Maltitol, Isomalt, Lactitol, Xylitol, Sorbitol, among others. Other examples of solids include polymers, such as polyethylene glycol, polyvinyl pyrrolidone, polyvinyl alcohol, among others. Other examples of solids include effervescent agents, such as sodium bicarbonate. The relevant physical properties of a solid that is bonded to a drug-containing fiber are high solubility and diffusivity in physiological/body fluids to ensure its rapid removal after contact with physiological/body fluid. Thus other non-limiting examples of a solid include solid active pharmaceutical ingredients with high solubility and diffusivity, such as Aliskiren. Typically, a solid material should have a solubility in physiological/body fluid under physiological conditions greater than 50 g/l to be removed or dissolved rapidly after contact with dissolution medium. This includes, but is not limited to a solubility greater than 75 g/l, or greater than 100 g/l, or greater than 150 g/l. The diffusivity of the solid material (as dissolved molecule in physiological/body fluid under physiological conditions) should typically be greater than 4×10⁻¹² m²/s if the solid material must be dissolved rapidly after contact with dissolution medium. This includes, but is not limited to a diffusivity greater than 6×10⁻¹² m²/s, or greater than 8×10⁻¹² m²/s, or greater than 1×10⁻¹¹ m²/s.

Furthermore, one or more filler materials such as microcrystalline cellulose or others, one or more sweeteners, one or more taste masking agents, one or more stabilizing agents, one or more preservatives, one or more coloring agents, or any other common or uncommon excipient may be added as excipient to the dosage form.

In some embodiments, a disintegration time of the dosage form (or the drug-containing solid) is no greater than (or less than) 45 minutes. This includes, but is not limited to a disintegration time no greater than 30 minutes, no greater than 25 minutes, no greater than 20 minutes, or no greater than 15 minutes. In the context of this disclosure, the disintegration time is defined as the time required to release 80 percent of the drug content of a representative dosage form structure into a stirred dissolution medium. The released drug may be a solid, such as a solid drug particle, and/or a molecule, such as a dissolved drug molecule. The disintegration test may, for example, be conducted with a USP disintegration apparatus under physiological conditions. (See, e.g. The United States Pharmacopeial Convention, USP 39-NF 34). Another method without limitation to conduct a disintegration test is by a USP basket apparatus (i.e., a USP apparatus 1 as shown in The United States Pharmacopeial Convention, USP 39-NF 34) under physiological conditions (e.g., at a temperature of 37° C. and at a stirring rate or basket rotation rate of 50-150 rpm). In this method, the time to disintegrate 80 percent of the representative dosage form structure after immersion in the stirred dissolution medium may, for example, be determined by visual or other optical methods. It may be noted that if the drug is in molecular form immediately or almost immediately after it is released from the dosage form structure, the disintegration time is about the same as the time to dissolve 80 percent of the drug content of a representative dosage form structure after immersion in a stirred dissolution medium. By way of example but not by way of limitation, the drug release time of dosage form structures comprising particles of highly water-soluble drugs in a water-soluble excipient matrix may be limited or determined by the disintegration time of the dosage form. As the dosage form structure disintegrates the released drug particles dissolve rapidly and the drug is in molecular form soon after the dosage form structure has disintegrated.

In case the drug-containing elements are well bonded to each other (or to a solid material that fills the one or more free spaces), the greater of a tensile strength or a yield strength of the assembled dosage form material is no less than 0.005 MPa. In other examples without limitation, the greater of a tensile strength or a yield strength of the assembled dosage form material is no less than 0.01 MPa, or 0.015 MPa, or 0.02 MPa, or 0.025 MPa, or 0.04 MPa, or 0.06 MPa, or 0.1 MPa, or 0.25 MPa, or 0.5 MPa. Bonding between the drug containing elements (or between the elements and the content of the free space) can, for example, be by interdiffusion of molecules, mechanical interlocking, or by other forces due to the surface energy of the materials. In some embodiments, good bonding is achieved without deforming the drug-containing elements plastically in the solid state. In this case, it may be possible to readily distinguish the elements from the free spaces in an image of the cross section of the dosage form (e.g., a scanning electron micrograph, a computerized tomograph, an x-ray image, or an image taken by another technique).

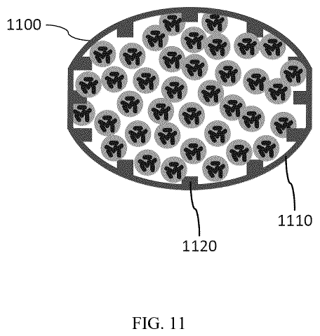

In some embodiments, the mechanical properties of the structural assembly of one or more elements disclosed herein (particularly the structures with weakly bonded elements (or segments), or even not bonded or unbonded elements (or segments)) may be improved, for example, by applying a coating on the surface of the dosage form (or the outer surface of the drug-containing solid). The thickness of the coating may, for example, be non-uniform. In the non-limiting example of FIG. 11, the coating 1100 comprises “thick” rings 1110 that provide mechanical support and “thin” sheets 1120 that disintegrate rapidly after the dosage form is immersed in a dissolution medium. In other non-limiting embodiments, the coating thickness may be uniform. We may note that a capsule encapsulating the dosage form (or the drug-containing solid) may also be considered a coating. Furthermore, it may be noted that in some embodiments of the invention disclosed herein, a coating may serve as taste masking agent, protective coating, means of providing color to the dosage form, enteric coating, means of improving the aesthetics of the dosage form, or have any other common or uncommon function of a coating. Moreover, in some non-limiting examples of the invention herein, a coating may be applied on the elements of the three dimensional structural network of elements.

Also the coating materials include, but are not limited to polyethylene glycol (PEG), polyvinylpyrrolidone (PVP), PEG-PVP copolymer, poloxamer, lauroyl macrogol-32 glycerides, polyvinylalcohol (PVA), PEG-PVA copolymer, polylactic acid, polyvinylacetate phthalate, polymethacrylates (e.g., poly (methacrylic acid, ethyl acrylate) 1:1, butylmethacrylat-(2-dimethylaminoethyl) methacrylat-methylmathacrylat-copolymer), gelatin, cellulose or cellulose derivatives (e.g., microcrystalline cellulose, hydroxypropyl cellulose, hydroxyethyl cellulose, methyl cellulose, hydroxypropyl methyl ether cellulose, hydroxypropyl methylcellulose), starch, polylactide-co-glycolide, polyvinyl caprolactam-polyvinyl acetate-polyethylene glycol graft copolymer, pregelatinized starch, lactose, sodium starch glycolate, or polyacrylic acid, Sucrose, Lactose, Maltose, Glucose, Maltodextrin, Mannitol, Maltitol, Isomalt, Lactitol, Xylitol, Sorbitol, a sweetener, a coloring agent, a preservative, a stabilizer, a taste masking agent, among others.

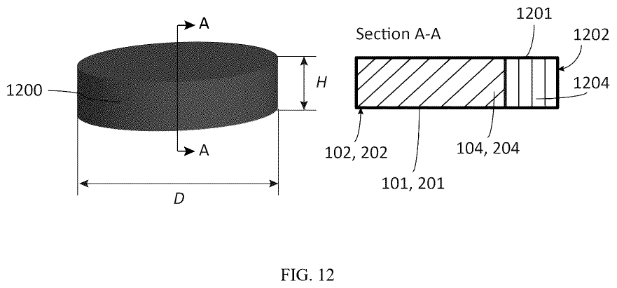

In some embodiments, in addition to the drug-containing solid 101, 201 described above, the dosage form 1200 disclosed herein may comprise another drug-containing solid 1201 that contains at least one active ingredient (or one or more other drug-containing solids that contain at least one active ingredient; all such other drug-containing solids are referred to here as “other solid” or “other drug-containing solid”). Said other drug-containing solid 1201 has an outer surface 1202 and internal structure 1204 contiguous with and terminating at said outer surface 1202 as shown in FIG. 12. In some embodiments, 80 percent of the other solid's 1201 drug content is converted to dissolved molecules in a time greater than 60 minutes after immersion of the dosage form in a physiological/body fluid under physiological conditions. In other embodiments, 80 percent of the other solid's 1201 drug content is converted to dissolved molecules in a time no greater than 60 minutes after immersion of the dosage form in a physiological/body fluid under physiological conditions.

### EXPERIMENTAL EXAMPLES

The following examples set forth, in detail, ways by which the structured dosage forms may be prepared and analyzed, and will enable one of skill in the art to more readily understand the principle thereof. The following examples are presented by way of illustration and are not meant to be limiting in any way.

### Example 1: Preparation of Melt-Processed Dosage Forms

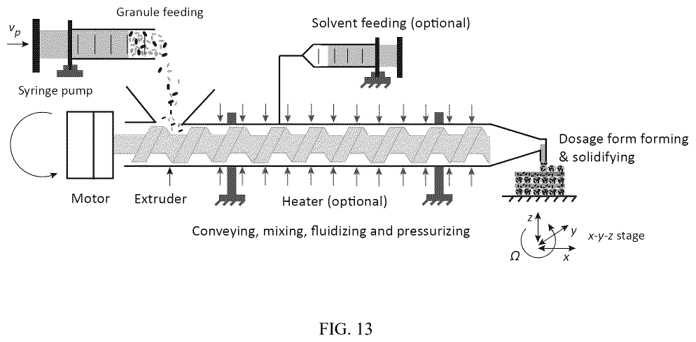

Melt-processed dosage forms were prepared by first mixing 40 wt % of solid Acetaminophen particles (particle size about 40-80 μm as received from Sigma, St. Louis) with 60 wt % granules of polyethylene glycol with a molecular weight of 35,000 g/mol (PEG 35k, as received from Sigma, St. Louis). The solid mixture was then loaded into the granule-feeding unit of an adapted extrusion-micropatterning machine as shown schematically in FIG. 13. The granule-feeding unit was set to deliver 1.7 mg/s of the drug and excipient material into the extruder barrel of the adapted extrusion-micropatterning machine. The rotation rate of the extruder screw was about 3-5 rpm and the temperatures of the extruder barrel and nozzle were set to 80° C.

Melt-processed fibrous dosage forms (e.g., structured dosage forms) were then micro-patterned as follows. A single-layer pattern of the fibrous effluent from the extruder nozzle was first deposited on a surface (also referred to herein as “moving platform” or “linear stage” or “x-y-z stage”) which was moved along the desired path in the x-y plane. It should be noted that in the invention herein, the terms “depositing on a surface”, “micropatterning”, “patterning”, “3D-micro-patterning”, “3D-printing”, etc. are used interchangeably. Further patterns were then added layer-by-layer to the deposited structure until the deposited fibrous structure reached the desired thickness. The velocity of the linear stage in the x-y plane was 7.3 mm/s during deposition (or patterning). The distance between the nozzle exit and the top of the deposited fibrous structure (or the deposition surface) was kept at 1 to 2 mm during patterning. The ambient temperature and that of the x-y-z stage were at room temperature. The process was stopped as soon as the thickness of the dosage form reached about 5 mm. Three different three dimensional structural networks of fibers were prepared: structures of the configuration shown in FIG. 1b with a nominal fiber radius, Rₙ=250 μm (equal to the inner radius of the extruder nozzle) and a nominal inter-fiber distance in a single layer, λₙ, of either 1750, 900, or 600 μm (λₙ is determined by the path of the x-y-z stage). In addition to the fibrous structures, single fibers of nominal radius, Rₙ=250 μm, were prepared by solidification of the fibrous effluent from the extruder nozzle. The position and velocity of the linear stage, and the velocity and radius (e.g. the half thickness) of the fibrous extrudate were precisely controlled during the process.

For preparing melt-processed minimally-porous solid dosage forms, a stainless steel mold was placed on the top surface of the linear stage and was filled with the effluent extrudate until a height of 5 mm was reached. The material was left in the mold, which was kept at room temperature, for about 2 minutes to solidify. Subsequently, the solid dosage form was ejected.

The elastic modulus and mechanical strength of the fibrous and minimally-porous dosage forms was sufficient to handle the dosage forms without taking specific care.

### Example 2: Preparation of Wet-Processed Dosage Forms

Wet-processed dosage forms were prepared by first mixing 60 wt % of solid ibuprofen particles (particle size about 20-40 μm, as received from BASF, Ludwigshafen, Germany) with 40 wt % particles of polyvinyl alcohol-polyethylene glycol graft copolymer 3:1 with a molecular weight of 45,000 Daltons (particle size about 50 μm, as received from BASF, Ludwigshafen, Germany; tradename: Kollicoat IR). The particles were mixed and loaded into the granule-feeding unit of an adapted extrusion-micropatterning machine as shown schematically in FIG. 13. The granule-feeding unit was set to deliver 1.7 mg/s (of the drug-excipient material) into the extruder barrel of the adapted extrusion-micropatterning machine. The liquid-feeding unit (e.g., the solvent-feeding unit) of the extrusion-micropatterning machine was filled with deionized water and set to deliver a water flow rate of 1.1 μl/s into the extruder barrel. The rotation rate of the extrusion screw was about 3-5 rpm.

For preparing wet-processed fibrous dosage forms (e.g., structured dosage forms), a single layer of the fibrous effluent from the extruder nozzle was micro-patterned on a moving surface (e.g., a “linear stage” or “x-y-z stage”) as described in the example 1 above. Then further patterns were added layer-by-layer to the deposited structure (e.g., the layers were stacked) until the deposited fibrous structure reached the desired thickness. The x-y-z stage (and the deposited structure) were position-and velocity-controlled. The velocity of the x-y-z stage was 14.4 mm/s during deposition of the material. The distance between the nozzle exit and the top of the fibrous structure (or the deposition surface defined by the x-y-z stage) was kept at 1 to 2 mm during patterning. The process was stopped when the thickness of the dosage form reached about 4-5 mm. After that warm air at a temperature of 60° C. was blown on the dosage form structure for about 5 minutes to dry the fibrous material. The fibrous dosage forms prepared had a three dimensional structural network of fibers as schematically shown in FIG. 1b. The nominal fiber radius of the dosage form structure, Rₙ=250 μm (as given by the inner radius of the extruder nozzle), and the nominal fiber-to-fiber spacing in a single layer, λₙ=900 μm. In addition to the fibrous dosage forms, single fibers were prepared by drying the fibrous effluent from the extruder nozzle as above.

For preparing wet-processed minimally-porous solid dosage forms, a stainless steel mold was placed on the top surface of the linear stage and was filled with the effluent stream until a height of about 4-5 mm was reached. The material was then left in the mold for about 48 hours in a dry environment at room temperature to remove the residual water. Subsequently, the dosage form was ejected from the mold.

### Example 3: Dosage Form Microstructures

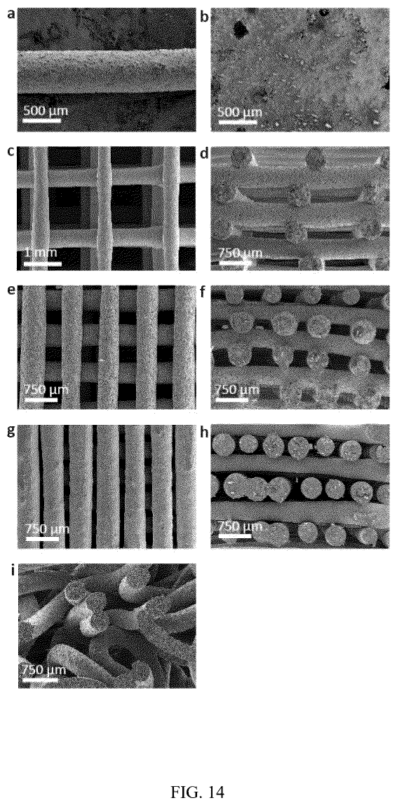

FIG. 14 presents scanning electron microscopy (SEM) images of example microstructures of melt-processed fibers and dosage forms. FIG. 14a is the image of a single fiber with drug particles embedded in an excipient matrix. The diameter of the fiber is roughly 539 μm (as listed in Table 1), slightly greater than the inner diameter of the nozzle exit. FIG. 14b is the microstructure of an essentially non-porous solid dosage form with drug particles embedded in an excipient matrix. FIGS. 14c-14h are microstructures of the fibrous dosage forms. The structures are ordered. Also, the fiber radius, R, and the inter-fiber distance, λ, are predictable and agree well with the nominal parameters set by the inner radius of the extruder nozzle and the path of the x-y-z stage as summarized in Table 1. FIG. 14i shows a wet-processed fibrous structure with random or almost random (e.g., not ordered) assembly/arrangement of the fibers. This structure was obtained if the distance between the nozzle exit and the top of the fibrous layer (or the deposition surface defined by the linear stage) was increased to about 15 mm.

### Example 4: Fiber and Dosage Form Disintegration

For imaging melt-processed dosage form and fiber disintegration, the dosage forms and the fiber were first attached to a sample holder using a drop of Loctite Super Glue. The sample holder was then immersed in the dissolution fluid which was a 0.05 M phosphate buffer solution (prepared with sodium phosphate monobasic and sodium phosphate dibasic) at a pH of 5.8 and at 37° C. (as suggested by the monograph of The United States Pharmacopeial Convention, USP 39-NF 34). For imaging dosage form disintegration, the fluid was stirred with a paddle rotating at 50 rpm during the entire dosage form disintegration time and images were captured at specific time points. Images of the disintegrating fibers were captured in both a stirred medium as above and also in a still (not stirred) dissolution fluid. All the images of dosage form disintegration were taken with a Nikon DX camera equipped with an additional 7 diopters of magnification.

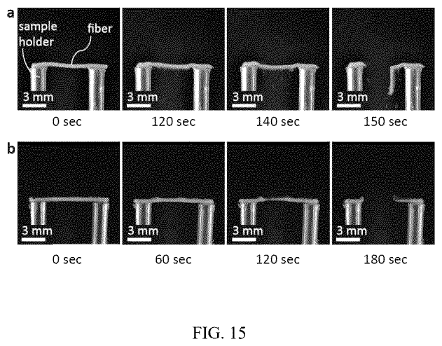

Representative images that present the disintegration processes of melt-processed single fibers (i.e., fibers consisting of 60% PEG 35k and 40% Acetaminophen) in still and stirred dissolution fluid are shown in FIG. 15. FIG. 15a is the series of images of a single fiber in stagnant (not stirred) medium. Soon after immersion of the fiber in the fluid, a viscous layer surrounding the fiber developed. The layer grew with time and starting at 60-90 seconds after immersion, small fragments fell downwards from the fiber (the density of the viscous layer (and the fiber) were slightly greater than that of water, and hence the viscous layer was flowing downwards due to gravity). Furthermore, at about 100-135 seconds after immersion, the fiber had deflected downwards by about 100-300 μm from its initial position. At a time t₁≈150 seconds, the fiber broke away from its support and fell down. (The fiber radius remained roughly constant during the entire process shown suggesting that the radial expansion due to fiber swelling is roughly compensated by the removal of material from the fiber into the dissolution fluid). An effective diffusivity of dissolution medium in a fiber may be estimated as D_{eff}=R₂/t₁=(269.5×10⁻⁶)²/150=4.8×10⁻¹⁰ m²/s (269.5 μm is the initial radius of a wet-processed fiber). Similarly, a rate of penetration of dissolution medium into a fiber is about R/t₁=269.5/150 μm/s=1.8 μm/s.

Images of the disintegration process of a melt-processed single fiber in stirred dissolution medium are shown in FIG. 15b. Here also a viscous layer that surrounded the fiber developed soon after immersion. But unlike in the previous case, the radius of the fiber decreased continuously with time until the fiber disappeared about 150 seconds after immersion (e.g., the viscous layer is continuously sheared away by convection in the stirred medium).

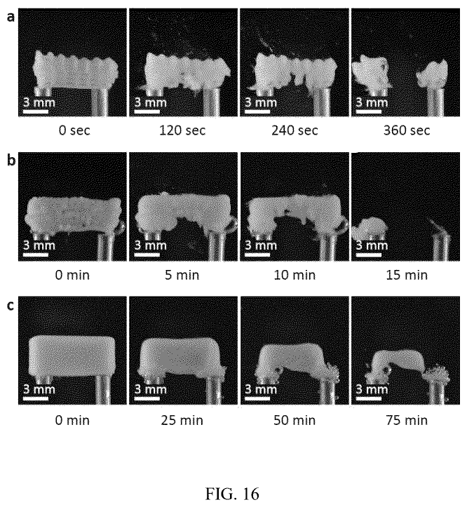

FIG. 16 presents selected images of melt-processed fibrous and non-porous dosage forms during disintegration in a stirred medium. The disintegration of a dosage form with R/λ=0.14 (i.e., a volume fraction of fibers, φ_{f}=0.22) is shown in FIG. 16a. Immediately after immersion in the dissolution medium, the void spaces (or free spaces) of the structure were filled with the fluid. The fibrous microstructure then started to transition from clear to diffuse. At the same time, fluidized material was removed from the structure until it finally disappeared. The disintegration time of the dosage form increased by about a factor of two compared with the single fiber. Images of the disintegration of a fibrous dosage form with R/λ=0.39 (φ_{f}=0.61) are presented in FIG. 16b. As in the previous case, after immersion of the dosage form, the void spaces were filled with fluid, the solid phase transitioned from clear to diffuse (e.g., from solid or solid-like to fluidic or fluid-like), and the fluidized material was then removed from the dosage form. The time to disintegrate the dosage form, however, increased by about a factor of 2-3 compared with the fibrous assembly with smaller R/λ (or φ_{f}) shown in FIG. 16a.

FIG. 16c illustrates disintegration of the melt-processed non-porous solid dosage form. The dosage form eroded continuously from the top and bottom surfaces. The disintegration time of this dosage form was more than a factor ten greater than that of the fibrous dosage form with φ_{f}=0.22.

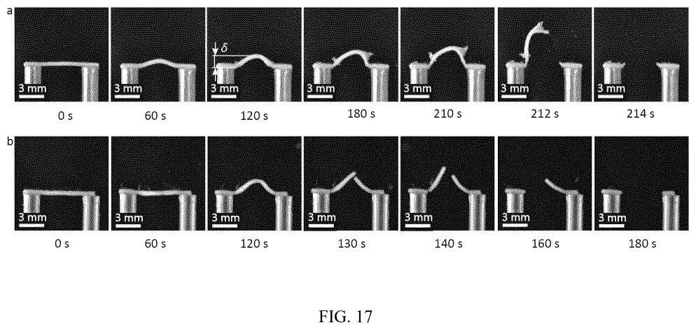

The disintegration process of a wet-processed single fiber (i.e., a fiber consisting of 60% ibuprofen and 40% polyvinyl alcohol-polyethylene glycol graft copolymer with molecular weight ˜45,000 Da) in still (i.e., unstirred) dissolution medium is shown in FIG. 17a. The dissolution fluid was a 0.05 M phosphate buffer solution (prepared with sodium phosphate monobasic and sodium phosphate dibasic) at a pH of 7.2 and at 37° C. Soon after immersion of the fiber in the fluid, a viscous layer surrounding the fiber developed. The viscous layer grew in thickness with time, and so did the fiber radius (and length, i.e., which is why the fibers bent and buckled with time). Some small fragments of the viscous layer, however, were removed from the fiber as the layer grew. Furthermore, starting at about 30-60 seconds after immersion, the fiber bent upwards and deflected from its initial position. The displacement increased with time. The maximum displacement, δ, reached about one third of the initial fiber length at a time t₁=120 seconds after immersion. Thus the fiber length at t₁=120 s is about 1/cos(atan(2/3))=1.2 times the initial fiber length. The fiber has therefore deformed substantially at this time and an effective diffusivity of dissolution medium in a fiber may be estimated as D_{eff}=R²/t₁=(200×10⁻⁶)²/120 m²/s=3.3×10⁻¹⁰ m²/s (200 μm is the initial radius of a wet-processed fiber). Similarly, a rate of penetration of dissolution medium into a fiber is about R/t₁=200/120 μm/s=1.7 μm/s.

FIG. 17b presents selected images of the disintegration of a wet-processed single fiber in stirred medium. Again, a viscous layer surrounding the fiber developed, and starting at 30-90 seconds after immersion the fiber bent upwards and deflected from its initial position. Then at about 130 seconds, the fiber broke in half, and at 140-180 seconds both halfs broke away from the support. The fiber radius decreased slightly within the experimental time frame. Thus more material was removed from the fiber in the stirred medium than in the unstirred medium.

Before the disintegration of wet-processed dosage forms was imaged, the edges of the dosage form were cut away so that the microstructural topology was uniform across the entire structure. Imaging of the disintegration of wet-processed dosage forms and fibers was done the same way as the melt-processed dosage forms.

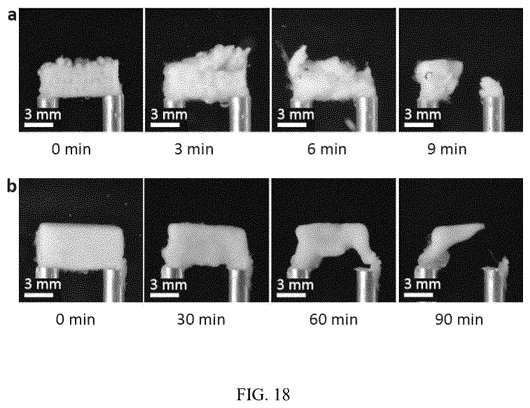

The disintegration of a wet-processed fibrous dosage form with R/λ=0.27 (i.e., a volume fraction of fibers, φ_{f}=0.42) is shown in FIG. 18a. Immediately after immersion in the dissolution medium, the void spaces (i.e., the free spaces) of the structure were filled with the fluid. The fibrous microstructure then started to transition from clear to diffuse (e.g., from solid or solid-like to fluidic or fluid-like). At the same time, fluidized material was removed from the structure. Furthermore, fibrous elements or small assemblies of such elements broke away from the dosage form until it disintegrated. The disintegration time of the dosage form was about a factor of 2-3 greater than that of the single fiber.

FIG. 18b illustrates disintegration of the wet-processed minimally-porous solid dosage form. The dosage form eroded continuously from the top and bottom surfaces. The disintegration time, however, increased by more than a factor of ten compared with the fibrous dosage form with φ_{f}=0.42.

### Example 5: Drug Release

Drug release (and drug dissolution) from fibers and dosage forms was tested by a USP dissolution apparatus 1 (as shown, e.g., in The United States Pharmacopeial Convention, USP 39-NF 34) filled with 900 ml of the dissolution fluids above (a 0.05 M phosphate buffer solution with pH 5.8 for melt-processed fibers and dosage forms and pH 7.2 for wet-processed fibers and dosage forms. The temperature of the dissolution fluid was 37±2° C. in both cases). The basket was rotated at 50 rpm. The concentration of dissolved drug in the dissolution fluid was measured versus time by UV absorption at 244 nm using a fiber optic probe. For all the dosage forms, the fraction of drug dissolved increased steadily with time at roughly constant rate until it plateaued out to the final value.

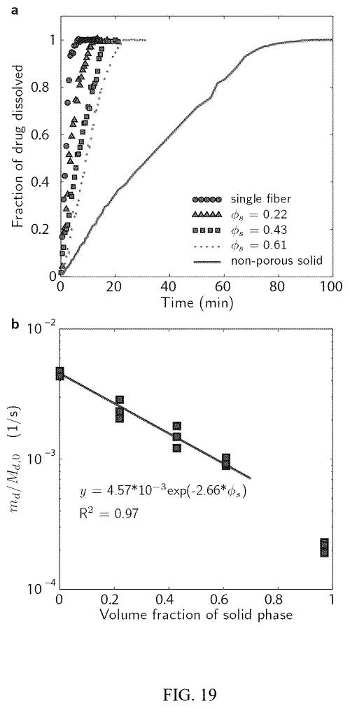

FIG. 19a presents representative curves of the fraction of drug dissolved versus time of the melt-processed fibrous dosage forms together with the data of a single fiber and the drug release results of the non-porous solid structure. The time to dissolve 80% of the drug content, t_{0.8}, can thus be readily extracted from these curves. The average t_{0.8} values of the various dosage forms tested are listed in Table 1. The average t_{0.8} of the single fiber is roughly 2.9 mins. t_{0.8} increases if the fibers are assembled to a dosage form and the volume fraction of solid is increased, to 5.64 mins for the dosage form with φ_{f}=0.22, and to about 14.2 mins if φ_{f}=0.61. t_{0.8} of the fibrous dosage forms, however, is much faster than the drug release time of the corresponding non-porous solid structure with t_{0.8}=63 mins.

FIG. 19b presents the fraction of drug dissolved per unit time, i.e., 1/M_{d,0}×dm_{d}/dt(=0.8/t_{0.8}), versus volume fraction of the solid phase (e.g., the volume fraction of fibers; M_{d,0} is the initial amount of drug in the dosage form, dm_{d}/dt the drug dissolution rate, and t_{0.8} the time to dissolve 80% of the drug content). The data of the fibrous dosage forms can be fitted to an exponential curve. The 1/M_{d,0}×dm_{d}/dt values of the solid dosage forms, however, do not follow this curve and are substantially smaller than predicted by the fit equation

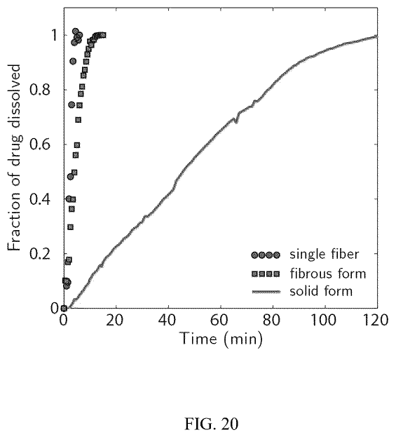

FIG. 20 shows representative curves of the fraction of drug dissolved versus time of the wet-processed fibrous dosage forms together with the data of a wet-processed

TABLE 1
Summary of microstructural parameters and drug dissolution times of single fibers, fibrous
dosage forms, and non-porous solid dosage forms.

| | 2Rₙ | 2R | λₙ | λ | | | | | t_{0.8} |
|---|---|---|---|---|---|---|---|---|---|
| | (μm) | (μm) | (μm) | (μm) | Rₙ/λₙ | R/λ | φ_{f,n} | φ_{f} | (min) |
| A | 500 | 539 | — | — | — | — | 0.0⁺ | 0.0⁺ | 2.89 |
| B | 500 | 490 ± 55 | 1750 | 1783 ± 47 | 0.14 | 0.14 | 0.22 | 0.22 | 5.64 |
| C | 500 | 505 ± 34 | 900 | 922 ± 38 | 0.28 | 0.27 | 0.44 | 0.43 | 9.14 |
| D | 500 | 485 ± 25 | 600 | 629 ± 70 | 0.42 | 0.39 | 0.65 | 0.61 | 14.17 |
| E | — | — | — | — | — | — | — | — | 63.00 |
| F | 500 | 408 ± 11 | 900 | — | — | — | 0.0⁺ | 0.0⁺ | 3.00 |
| G | 500 | 404 ± 68 | 900 | 745 ± 76 | 0.31 | 0.27 | 0.49 | 0.42 | 7.00 |
| H | 500 | — | — | — | — | — | — | — | 79.00 |

A: melt-processed single fiber; B, C, D: melt-processed fibrous dosage forms; E: melt-processed minimally-porous solid dosage form; F: wet-processed single fiber; G: wet-processed fibrous dosage form; H: wet-processed minimally-porous solid dosage form
The nominal fiber radius, Rₙ, is the inner diameter of extruder nozzle.
The nominal fiber-to-fiber distance, λₙ, is determined by the path along which the fiber is deposited.
The measured fiber radius, R, and fiber-to-fiber distance, λ, are obtained from SEM images of the cross section of the dosage form. Non-limiting examples of such images are shown in FIG. 14.
t_{0.8} is the time to dissolve 80% of the drug contained in the dosage form. It is derived from the results of drug release experiments shown in FIGS. 19 and 20.
The volume fraction of fibers in the fibrous dosage forms was calculated as φ_{f} = πR/2A.

single fiber and the drug release results of wet-processed minimally-porous solid structure. The time to dissolve 80% of the drug content, t_{0.8}, is readily extracted from these curves. The average t_{0.8} values of the various dosage forms tested are listed in Table 1. The average t_{0.8} of the single fiber is roughly 3 mins. t_{0.8} increases if the fibers are assembled to a dosage form, to 7 mins. t_{0.8} of the fibrous dosage forms, however, is much faster than the drug release time of the corresponding minimally-porous solid structure with t_{0.8}=79 mins.

### Example 6: Viscosities of PEG 35k-Water Solutions

The shear viscosities of PEG 35k-physiological/body fluid solutions was determined by first mixing water with PEG 35k at a polymer concentration of 5, 10, 20, 33, and 47 wt % (i.e., the water weight fractions were 95, 90, 80, 67, and 53 wt %). A shear rheometer (TA Instruments, ARG2 Rheometer, stress-controlled) equipped with a 60 mm diameter cone with an apex angle of 178° was used. The temperature was 37±1° C. during the experiments, and the shear strain-rate range was 1-100/s.

It was found that the shear viscosities of the PEG 35k-water solutions investigated were highly dependent on the weight fraction of polymer, wₚ, in the measured range 0.05<wₚ<0.47. But the shear viscosity of a specific solution (e.g., a solution at a given weight fraction of the polymer) was mostly independent of shear rate \dot{γ}, if 1 s⁻¹<\dot{γ}<100 s⁻¹. In FIG. 21, an average of the viscosity measured in the given shear rate range is plotted versus the polymer weight fraction. At small weight fractions of the polymer (i.e. in the range 0.05<wₚ<0.16), the shear viscosity, μₛ, follows roughly μₛ=0.314×wₚ^{1.31}. As the weight fraction of polymer is increased beyond about 0.15, however, the curve of μₛ versus wₚ changes to a much stronger dependence on wₚ. The viscosity roughly follows μₛ=194×wₚ^{4.79} if 0.16<wₚ<0.47.

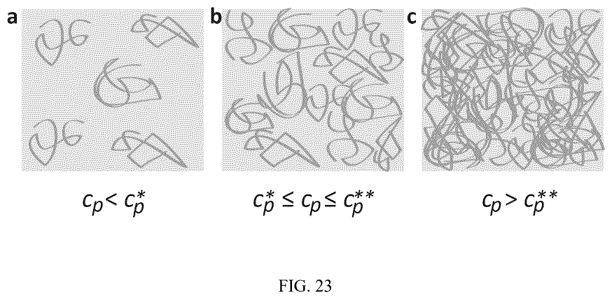

From this data, both the microstructure and select properties of the polymer solution can be estimated. In an infinitely dilute water-polymer solution where the polymer molecules can be considered as individual units that do not touch as shown later in FIG. 23a, according to the Einstein viscosity relation, the solution viscosity is a linear function of the polymer concentration, cₚ. The experimental results suggest that the dilute solution approximations are valid in the range 0.05<wₚ<0.16.

In a dilute solution, the diffusivity, Dₚ, of a linear polymer (a relevant property for dosage form disintegration) in a θ-solvent follows Zimm's equation:

$${D}_{p} = 0.192 \frac{{k}_{b} T}{{N}^{05} b {μ}_{l}} \qquad ( 17 )$$

where k_{b} is Boltzmann's constant, T the temperature, N the number of bonds of the polymeric chain, and b the bond length. Using T=310 K, N=2385, b=1.54 A, and μₗ=0.001 Pa·s (as for the dilute PEG 35k-water solutions), Dₚ=1.09×10⁻¹⁰ m²/s.

The dilute solution assumptions, however, break down if the concentration is increased to the value where the molecules touch. The critical polymer concentration, cₚ*, at which the polymer molecules entangle (another relevant property for dosage form disintegration) is about:

$${c}_{p}^{*} ≅ \frac{3 M}{4 π {N}_{A} {R}_{g}^{3}} = \frac{3 M}{4 π {N}_{A}} {( \frac{\sqrt{6}}{{N}^{v} b} )}^{3} \qquad ( 18 )$$

where M is the molecular weight of the polymer and N_{A} Avogadro's number. The experimental data presented in FIG. 21 suggest that wₚ*≈0.16 and cₚ*≈163.4 kg/m³. This result agrees with the value calculated by Eq. (18) if the parameter values given above are used and the Flory exponent v=0.55.

The solution is considered semi-dilute if the polymer concentration is above cₚ*. Typically, in the semi-dilute region μₛ≈cₚ⁴⁻⁶. This law is in agreement with our experiments in the range 0.16<wₚ<0.47.

### Example 7: Viscosities of Kollicoat IR-Water Solutions

The shear viscosities of Kollicoat IR-physiological/body fluid solutions were determined by first mixing water with Kollicoat IR at polymer concentrations of 2.5, 5, 10, 15, 20, 25, 30, 35, and 40 wt %. A shear rheometer (TA Instruments, ARG2 Rheometer, stress-controlled) equipped with a 60 mm diameter cone with an apex angle of 178° was used. The temperature was 37° C. during the experiments, and the shear strain-rate range was 1 s⁻¹-100 s⁻¹.

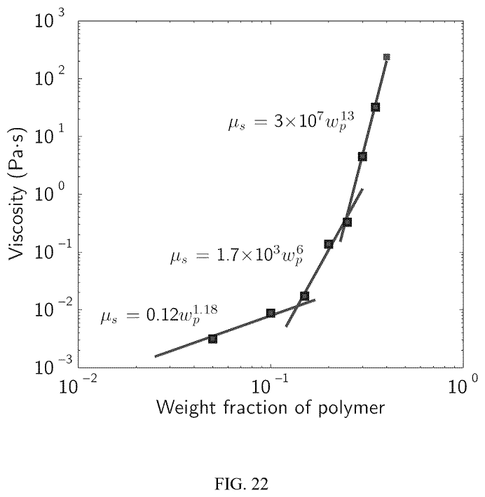

FIG. 22 presents the viscosity versus weight fraction of polymer. At small weight fractions of the polymer (i.e. in the range 0.025<wₚ<0.14), the shear viscosity follows roughly μₛ=0.12×wₚ^{1.18}. Then if 0.14<wₚ<0.25, the shear viscosity is about μₛ=1.7×wₚ⁶, a much stronger dependence on wₚ. As the weight fraction of polymer is increased beyond 0.25, the curve of μₛ versus wₚ changes to an even stronger dependence on wₚ. In the range 0.25<wₚ<0.4 the viscosity roughly follows μₛ=3×10⁷wₚ¹³.

These results allow us to estimate the structure of the water-polymer solutions. The viscosity of an infinitely dilute water-polymer solution is a linear function of the polymer concentration, and the results of this work suggest that the solution is dilute up to wₚ*=0.14. In such a dilute solution, the polymer molecules are individual molecules surrounded by water. They do not touch each other or form an interconnected structure as shown in FIG. 23a.

Then in the semi-dilute region the solution viscosity typically follows μₛ˜cₚ⁴⁻⁶. This work suggests that the solution is semidilute if 0.14<wₚ<0.25. In such semidilute solutions, the polymer molecules touch, but entanglement of the chains of different molecules is minimal (FIG. 23b).

If the polymer concentration is increased beyond wₚ**(or cₚ**) (wₚ**≈0.25 in the system of this example), however, the polymer molecules may entangle to fit in the given space (FIG. 23c). This results in stronger dependence of shear viscosity versus weight fraction of polymer. In the system of the present example, μₛ˜wₚ¹³ in this region. The solution is therefore concentrated. We may note that well within the concentrated region, at a polymer concentration of 0.35 or above, the material may behave like a semisolid.

### Dosage Form Application Examples

In some embodiments, the amount of active ingredient contained in a dosage form disclosed in this invention is appropriate for administration in a therapeutic regimen that shows a statistically significant probability of achieving a predetermined therapeutic effect when administered to a relevant population. By way of example but not by way of limitation, active ingredients may be selected from the group consisting of acetaminophen, aspirin, caffeine, ibuprofen, an analgesic, an anti-inflammatory agent, an anthelmintic, anti-arrhythmic, antibiotic, anticoagulant, antidepressant, antidiabetic, antiepileptic, antihistamine, antihypertensive, antimuscarinic, antimycobacterial, antineoplastic, immunosuppressant, antihyroid, antiviral, anxiolytic and sedatives, beta-adrenoceptor blocking agents, cardiac inotropic agent, corticosteroid, cough suppressant, diuretic, dopaminergic, immunological agent, lipid regulating agent, muscle relaxant, parasympathomimetic, parathyroid, calcitonin and biphosphonates, prostaglandin, radiopharmaceutical, anti-allergic agent, sympathomimetic, thyroid agent, PDE IV inhibitor, CSBP/RK/p38 inhibitor, or a vasodilator).

In conclusion, this invention discloses a dosage form with predictable structure and drug release behaviour. Both can be tailored by well-controllable parameters. This enables faster and more economical development and manufacture of pharmaceutical dosage forms, and higher quality and more personalized medical treatments.

It is contemplated that a particular feature described either individually or as part of an embodiment in this disclosure can be combined with other individually described features, or parts of other embodiments, even if the other features and embodiments make no mention of the particular feature. Thus, the invention herein extends to such specific combinations not already described. Furthermore, the drawings and embodiments of the invention herein have been presented as examples, and not as limitations. Thus, it is to be understood that the invention herein is not limited to these precise embodiments. Other embodiments apparent to those of ordinary skill in the art are within the scope of what is claimed.

We claim:

1. A pharmaceutical solid dosage form comprising:
drug-containing solid having an outer surface and an internal structure contiguous with and terminating at said outer surface;
said internal structure comprising a continuous structural assembly of one or more repeatably arranged, extruded structural elements;
said extruded structural elements comprising at least one pharmaceutically active ingredient and at least a hydrophilic excipient;
said extruded structural elements further comprising segments separated and spaced from adjoining segments by free spacings, said free spacings definining one or more substantially interconnected free spaces through the drug-containing solid;
wherein
the one or more extruded structural elements are so arranged that an average ‘free spacing’ between segments is greater than 1 μm; and
upon immersion of said drug-containing solid in a physiological fluid under physiological conditions, said drug-containing solid comprises a disintegration time of less than 45 minutes.

2. The dosage form of claim 1, wherein average thickness of the one or more elements is no greater than 1 mm.

3. The dosage form of claim 1, wherein average thickness of the one or more elements is in the range between 5 μm and 2 mm.

4. The dosage form of claim 1, wherein the thickness of the one or more elements is precisely controlled.

5. The dosage form of claim 1, wherein average ‘free spacing’ between segments is in the range between 1 μm and 5 mm.

6. The dosage form of claim 1, wherein the ‘free spacing’ between segments of the one or more elements is precisely controlled.

7. The dosage form of claim 1, wherein at least one structural element comprises a fiber.

8. The dosage form of claim 1, wherein at least one structural element comprises a sheet.

9. The dosage form of claim 1, wherein at least one structural element comprises a bead that is bonded to another structural element.

10. The dosage form of claim 1, wherein upon immersion of the drug-containing solid in a physiological fluid under physiological conditions, said physiological or body fluid percolates a substantially interconnected free space.

11. The dosage form of claim 1, wherein no more than 15 walls must be ruptured to obtain an interconnected, continuous free space from a surface of the drug-containing solid to any point in the interior.

12. The dosage form of claim 1, wherein the free space is contiguous.

13. The dosage form of claim 1, wherein at least a structural element or a segment thereof is bonded to another structural element or another segment of said structural element.

14. The dosage form of claim 1, wherein at least a structural element or a segment thereof is bonded to another structural element or another segment of said structural element by solidification of a fluidic contact between said elements or segments.

15. The dosage form of claim 1, wherein at least one excipient comprises a solubility no less than about 1 g/l in a physiological or body fluid under physiological conditions.

16. The dosage form of claim 1, wherein at least one excipient comprises a solubility no less than about 10 g/l in a physiological or body fluid under physiological conditions.

17. The dosage form of claim 1, wherein at least one excipient is swellable by a body fluid, and wherein an effective diffusivity of water in said swellable excipient is greater than 1×10⁻¹¹ m²/s.

18. The dosage form of claim 17, wherein the swellable excipient comprises a viscosity less than 500 Pa·s upon absorption of a physiological fluid under physiological conditions.

19. The dosage form of claim 1, wherein at least one hydrophilic excipient is selected from the group comprising polyethylene glycol (PEG), polyethylene oxide, polyvinylpyrrolidone (PVP), PEG-PVP copolymer, poloxamer, lauroyl macrogol-32 glycerides, polyvinylalcohol (PVA), PEG-PVA copolymer, polylactic acid, polyvinylacetate phthalate, polymethacrylates (e.g., poly (methacrylic acid, ethyl acrylate) 1:1, or butylmethacrylat-(2-dimethylaminoethyl) methacrylat-methylmathacrylat-copolymer), gelatin, cellulose or cellulose derivatives (e.g., microcrystalline cellulose, hydroxypropyl cellulose, hydroxyethyl cellulose, methyl cellulose, hydroxypropyl methyl ether cellulose, or hydroxypropyl methylcellulose), starch, polylactide-co-glycolide, polyvinyl caprolactam-polyvinyl acetate-polyethylene glycol graft copolymer, lactose, starch derivatives (e.g., pregelatinized starch or sodium starch glycolate), chitosan, pectin, acrylic acid crosslinked with allyl sucrose or allyl pentaerythritol (e.g., carbopol), and polyacrylic acid.

20. The dosage form of claim 1, wherein a free space comprises a matter selected from the group comprising gas, liquid, or solid, and wherein said matter is partially or entirely removed upon contact with a physiological or body fluid under physiological conditions.
